(19) United States
(12) Patent Application Publication
(10) Pub. No.: US 2021/0163593 A1
(43) Pub. Date: Jun. 3, 2021

(54) TREATMENT OF MONOGENIC DISEASES WITH AN ANTI-CD45RC ANTIBODY

(71) Applicants: INSERM (INSTITUT NATIONAL DE LA SANTÉ ET DE LA RECHERCHE MÉDICALE), Paris Cedex 13 (FR); CENTRE HOSPITALIER UNIVERSITAIRE DE NANTES, Nantes Cedex 1 (FR); UNIVERSITÉ DE NANTES, Nantes (FR)

(72) Inventors: Carole GUILLONNEAU, La Chevrolière (FR); Ignacio ANEGON, Nantes (FR)

(73) Assignees: INSERM (INSTITUT NATIONAL DE LA SANTÉ ET DE LA RECHERCHE MÉDICALE), Paris Cedex 13 (FR); CENTRE HOSPITALIER UNIVERSITAIRE DE NANTES, Nantes Cedex 1 (FR); UNIVERSITÉ DE NANTES, Nantes (FR)

(21) Appl. No.: 16/772,917

(22) PCT Filed: Dec. 14, 2018
(86) PCT No.: PCT/EP2018/085016

§ 371 Date: Jun. 15, 2020

(30) Foreign Application Priority Data
Dec. 15, 2017 (EP) 17306783.6

(51) Int. Cl. C07K 16/28; A61K 45/06; A61K 47/68; A61P 43/00

(52) U.S. Cl. CPC C07K 16/289; A61K 45/06; A61K 2039/505; A61P 43/00; A61K 47/6801

(57) ABSTRACT

Anti-CD45RC antibodies, for use in the treatment of monogenic diseases caused by genes not associated with immune function but whose deficiency is associated with inflammation and/or immune reactions (such as genes deficient in Duchenne muscular dystrophy (DMD), cystic fibrosis, lysosomal diseases and al-anti-trypsin deficiency); or caused by genes involved in the immune system and whose deficiency generates inflammation and/or autoimmune reactions (such as genes deficient in T-cell primary immunodeficiencies such as IPEX (immunodysregulation polyendocrinopathy enteropathy X-linked syndrome), APECED (autoimmune polyendocrinopathy-candidiasis-ectodermal dystrophy), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome).

### FIELD OF INVENTION

The present invention relates to the use of anti-CD45RC antibodies, for preventing and/or treating monogenic diseases such as Duchenne muscular dystrophy (DMD) or autoimmune polyendocrinopathy-candidiasis-ectodermal dystrophy (APECED), and associated symptom.

### BACKGROUND OF INVENTION

Monogenic diseases are caused by single-gene defects. Over 4000 human diseases are caused by these defects linked to one particular gene. Up to now, most treatment options revolve around treating the symptoms of the disorders, in an attempt to improve patient quality of life. Gene therapy, i.e., a form of treatment where a healthy gene is introduced into the body of a patient, is the main hope for durable treatments of this type of diseases. However, major obstacles have been encountered during the development of techniques for the delivery of genes to the appropriate cells affected by the disorder.

Among monogenic diseases, some are linked to genes involved in the immune system and whose deficiency generates inflammation and/or autoimmune reactions. An example of such disease is the autoimmune polyendocrinopathy-candidiasis-ectodermal dystrophy (APECED). Other monogenic diseases are linked to genes not associated with immune functions but whose deficiency is associated with inflammation and/or immune reactions. An example of such disease is Duchenne muscular dystrophy (DMD).

DMD is a monogenic disease wherein mutations of the DMD gene coding for the protein dystrophin lead to severe X-linked muscular dystrophy, which affects all voluntary muscles, as well as the heart and breathing muscles in later stages.

Immune responses are involved in the pathophysiology of disease in both DMD patients and mdx mice which have the same dystrophin mutation as human patients (for a review, see Rosenberg et al., 2015. Sci Transl Med. 7(299):299rv4). Standard DMD patients management make use of corticoids, such as prednisolone. In mdx mice, treatments decreasing effector immune responses or inflammation, such as intravenous immunoglobulins, tranilast, heme oxygenase-1 inducers, IL-1 receptor antagonist and IL-2 to amplify regulatory T cells (T_{regs}) have also been used (Rosenberg et al., 2015. Sci Transl Med. 7(299):299rv4; Villalta et al., 2014. Sci Transl Med. 6(258):258ra142).

However, despite recent promising new treatments, the average life expectancy of DMD patients is still severely reduced. Hence, there remains a need for a treatment capable of reducing or inhibiting immune responses in DMD and in related diseases.

Autoimmune diseases are caused by breakdown of self-tolerance that leads to a dysfunction of the immune system and is the cause of serious, disabling and even fatal consequences. A better knowledge of immunological mechanisms involved in tolerance represents a major challenge to improve both the understanding and treatment of autoimmune diseases.

A key player in this equilibrium is AIRE, a transcription regulator that allows the expression of tissue-restricted antigens (TRA) in medullary epithelial thymic cells (mTECs) and auto-reactive T cells deletion.

The autoimmune polyendocrinopathy-candidiasis-ectodermal dystrophy (APECED), also known as auto-immune polyglandular syndrome type I (APS 1), is a rare multi-organ autosomal recessive autoimmune disease caused by mutations in the AIRE gene.

In human, this gene is located on locus 21q22.3 and more than 100 mutations have been described to cause APECED with a prevalence of 1-9:1000000 (Orphanet, http://www.orpha.net), this prevalence being increased in certain populations, such as Finnish, Norwegian, Sardinian and Iranian Jews. Among Sardinians for example, the over-representation of the R139X mutation is present in 90% of APECED patients.

The clinical phenotype of APECED is usually defined by the presence of 2 out of the 3 major symptoms: hypoparathyroidism, adrenal insufficiency (Addison's disease) and chronical muco-cutaneous candidiasis (CMC). This disease is also associated with multiple autoimmune and ectodermal features, such as type 1 diabetes, enamel hypoplasia, vitiligo, premature ovarian failure, keratitis, pernicious anemia, alopecia, exocrine pancreatitis, interstitial lung disease, nephritis and other disorders.

### SUMMARY

Herein, the Inventors have demonstrated that treatment of Dmd^{mdx} rats with anti-CD45RC antibodies could be used to treat DMD by specifically acting on cells expressing CD45RC (herein designated as CD45RC⁺ cells), and in particular, by acting on cells expressing high levels of CD45RC (herein designed as CD45RC^{high} cells or CD45RC+^{high} cells).

Moreover, the Inventors have showed that administration to Aire^{-/-} (KO) rats of an anti-CD45RC monoclonal antibody results in a strong depletion of CD45RC^{high} T lymphocytes, and to the inhibition of symptoms characteristics of APECED.

The present invention relates to an anti-CD45RC antibody, for use in the prevention and/or treatment of monogenic diseases selected from the group comprising:
monogenic diseases caused by a gene which is not associated with immune function but whose deficiency is associated with inflammation and/or immune reactions, and selected from Duchenne muscular dystrophy (DMD), cystic fibrosis, lysosomal diseases and α1-anti-trypsin deficiency; and/or
monogenic diseases caused by a gene involved in the immune system and whose deficiency generates inflammation and/or autoimmune reactions, and selected from immunodysregulation polyendocrinopathy enteropathy X-linked syndrome (IPEX), autoimmune polyendocrinopathy-candidiasis-ectodermal dystrophy (APECED), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

In some embodiments, said anti-CD45RC antibody is a monoclonal antibody.

In some embodiments, said anti-CD45RC antibody is an anti-human CD45RC monoclonal antibody.

In some embodiments, said anti-CD45RC antibody is a chimeric antibody, a bispecific antibody, a humanized antibody or a fully human antibody.

In some embodiments, prevention and/or treatment of monogenic diseases comprises reduction, alleviation, lessening and/or inhibition of symptoms or signs associated with said monogenic diseases, preferably of autoimmune and/or inflammatory symptoms or signs.

In some embodiments, said anti-CD45RC antibody depletes T CD45RChigh cells.

In some embodiments, said anti-CD45RC antibody is a multispecific antibody comprising a first antigen binding site directed against CDR45RC and at least one second antigen binding site directed against an effector cell able to mediate depletion of T CD45RChigh cells through direct binding, antibody-dependent cell-mediated cytotoxicity (ADCC), complement dependent cytotoxicity (CDC), and/or antibody-dependent phagocytosis.

In some embodiments, said anti-CD45RC antibody is conjugated to a cytotoxic moiety.

In some embodiments, said anti-CD45RC antibody is in the form of a pharmaceutical composition comprising said anti-CD45RC antibody and a pharmaceutically acceptable carrier or excipient or vehicle.

In some embodiments, said anti-CD45RC antibody is to be administered in combination with an immunosuppressive and/or anti-inflammatory drug.

In some embodiments, said anti-CD45RC antibody is to be administered in combination with gene therapy or cell therapy.

In some embodiments, said gene therapy or cell therapy is to be administered before or after administration of said anti-CD45RC antibody, preferentially before administration of said anti-CD45RC antibody.

In some embodiments, said monogenic disease is selected from DMD, cystic fibrosis, lysosomal storage diseases and al-anti-trypsin deficiency, preferably said monogenic disease is DMD.

In some embodiments, said monogenic disease is selected from IPEX, APECED, B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome, preferably said monogenic disease is APECED.

Definitions

“Antibody” or “Immunoglobulin”

As used herein, the terms “antibody” and “immunoglobulin” refer to a protein having a combination of two heavy and two light chains whether or not it possesses any relevant specific immunoreactivity. “Antibodies” refers to such assemblies which have significant known specific immunoreactive activity to an antigen of interest (e.g., SEQ ID NO: 1). The term “anti-CD45RC antibodies” is used herein to refer to antibodies which exhibit immunological specificity for human CD45RC protein. As explained elsewhere herein, “specificity” for CD45RC does not exclude cross-reaction with species homologues of CD45RC.

The terms “antibody” and “immunoglobulin”, as used herein, are also meant to encompass antibody binding fragments.

It will also be appreciated that the terms “antibody” and “immunoglobulin”, as used herein, encompass modified antibodies or antibody binding fragments, using known methods. For example, to slow clearance in vivo and obtain a more desirable pharmacokinetic profile, an antibody or binding fragment thereof may be modified with polyethylene glycol (PEG). Methods for coupling and site-specifically conjugating PEG to an antibody or binding fragment thereof are described in, e.g., Leong et al., 2001. Cytokine. 16(3):106-19; Delgado et al., 1996. Br J Cancer. 73(2):175-82.

Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (V_{L} domain) and heavy chain variable domain (VH domain) of an antibody combine to form the variable region that defines a three-dimensional antigen binding site. This quaternary antibody structure forms the antigen binding site presents at the end of each arm of the “Y”. More specifically, the antigen binding site is defined by three complementarity determining regions (CDRs) on each of the V_{H} and V_{L} chains.

“Antibody Binding Fragments”

The term “antibody binding fragment”, as used herein, refers to a part or region of an antibody, which comprises fewer amino acid residues than the whole antibody. A “binding fragment” binds an antigen (e.g., SEQ ID NO: 1) and/or competes with the whole antibody from which it was derived for antigen binding. Antibody binding fragments encompasses, without any limitation, single-chain antibodies, dimeric single chain antibodies, single-domain antibodies, Fv, Fab, Fab′, Fab′-SH, F(ab)′2, Fd, defucosylated antibodies, bi-specific antibodies, diabodies, triabodies and tetrabodies, just to name a few.

A “single chain antibody” refers to any antibody or fragment thereof that is a protein having a primary structure comprising or consisting of one uninterrupted sequence of contiguous amino acid residues, including without limitation (1) single-chain Fv molecules (scFv); (2) single chain proteins containing only one light chain variable domain, or a fragment thereof that contains the three CDRs of the light chain variable domain, without an associated heavy chain moiety; and (3) single chain proteins containing only one heavy chain variable region, or a fragment thereof containing the three CDRs of the heavy chain variable region, without an associated light chain moiety.

A “single-chain Fv”, also abbreviated as “sFv” or “scFv”, refers to antibody fragments that comprise the V_{H} and V_{L} antibody domains connected into a single amino acid chain. Preferably, the scFv amino acid sequence further comprises a peptide linker between the V_{H} and V_{L} domains that enables the scFv to form the desired structure for antigen binding (Plückthun, 1994. Antibodies from Escherichia coli. In Rosenberg & Moore (Eds.), The pharmacology of monoclonal antibodies. Handbook of Experimental Pharmacology, 113:269-315. Springer: Berlin, Heidelberg).

An “Fv” refers to the minimum antibody fragment that contains a complete antigen-recognition and -binding site. This fragment consists of a dimer of one HCVR and one LCVR in tight, non-covalent association. From the folding of these two domains emanate six hypervariable loops (three loops each from the heavy and light chain) that contribute to antigen binding and confer antigen binding specificity to the antibody. However, even a single variable domain (or half of an Fv comprising only three CDRs specific for an antigen) has the ability to recognize and bind antigen, although at a lower affinity than the entire binding site.

A “diabody” refers to a small antibody fragment prepared by constructing scFv fragments with short linkers (about 5-10 residues) between the HCVR and LCVR such that inter-chain but not intra-chain pairing of the variable domains is achieved, resulting in a bivalent fragment, i.e., fragment having two antigen-binding sites. Bispecific diabodies are heterodimers of two crossover scFv fragments in which the HCVR and LCVR of the two antibodies are present on different polypeptide chains. Diabodies are described more fully in Patent EP0404097, Patent application WO1993011161; and Holliger et al., 1993. Proc Natl Acad Sci USA. 90(14):6444-8.

Antibody binding fragments can be obtained using standard methods. For instance, Fab or F(ab′)2 fragments may be produced by protease digestion of the isolated antibodies, according to conventional techniques.

“Antibody-Dependent Cell-Mediated Cytotoxicity” or “ADCC”

As used herein, the terms “antibody-dependent cell-mediated cytotoxicity” or “ADCC” refer to a form of cytotoxicity in which secreted antibodies bound onto Fc receptors (FcRs) present on certain cytotoxic cells (e.g., NK cells, neutrophils, monocytes and macrophages) enable these cytotoxic effector cells to bind specifically to an antigen-bearing target cell and subsequently to kill the target cell. To assess ADCC activity of a molecule of interest, an in vitro ADCC assay, such as that described in Patents U.S. Pat. Nos. 5,500,362 or 5,821,337, may be performed.

“Antibody-Dependent Phagocytosis” or “Opsonization”

As used herein, the terms “antibody-dependent phagocytosis” or“opsonization” refer to the cell-mediated reaction wherein non-specific cytotoxic cells that express FcγRs recognize bound antibody on a target cell and subsequently cause phagocytosis of the target cell.

“CD45”

As used herein, the term “CD45” (also known as CD45R or PTPRC) refers to a transmembrane glycoprotein existing in different isoforms. These distinct isoforms of CD45 differ in their extracellular domain structures which arise from alternative splicing of 3 variable exons (exons 4, 5 and 6) coding for the A, B and C determinants, respectively, of the CD45 extracellular region. The various isoforms of CD45 have different extracellular domains, but have an identical extracellular sequence proximal to the membrane, as well as for the transmembrane domain and a large cytoplasmic tail segments containing two tandemly homologous highly conserved phosphatase domains of approximately 300 residues. CD45 and its isoforms non-covalently associate with lymphocyte phosphatase-associated phosphoprotein (LPAP) on T and B lymphocytes. CD45 has been reported to be associated with several other cell surface antigens, including CD1, CD2, CD3, and CD4. CD45 is involved in signaling lymphocytes activation.

“CD45RC”

As used herein, the term “CD45RC” refers to a 200-220 kDa single chain type I membrane glycoprotein well-known from the skilled artisan. CD45RC is an alternative splicing isoform of CD45 comprising exon 6 encoding the C determinant (hence the terminology CD45RC, i.e., CD45 Restricted to the C determinant), but lacking exons 4 and 5, respectively encoding the A and B determinants This CD45RC isoform is expressed on B cells, and a subset of CD8⁺ T cells and CD4⁺ T cells, but not on CD8⁺ or CD4⁺ Treg, CD14⁺ monocytes or PMN (Picarda et al., 2017. JCI Insight. 2(3):e90088). While some monoclonal antibodies can recognize an epitope in the portion of CD45 common to all the different isoforms (these are termed anti-CD45 antibodies), other monoclonal antibodies have restricted specificity to a given isoform, depending on which determinant they recognize (A, B or C).

“CD45RC⁺ Cell Antigen” or “CD45RC⁺ Cell Surface Marker”

As used herein, the terms “CD45RC⁺ cell antigen” or “CD45RC⁺ cell surface marker” refer to an antigen (or epitope) of sequence SEQ ID NO: 1, which is expressed or displayed at the surface of a CD45RC⁺ cells (including T cells, B cells and natural killer (NK) cells) which can be targeted with an anti-CD45RC agent which binds thereto (such as an antibody or an aptamer). Exemplary CD45RC⁺ T cell surface markers include but are not limited to the CD45RC as previously described or other antigens that characterize said population of T cells. The CD45RC⁺ T cells surface marker of particular interest is preferentially expressed on CD45RC⁺ T cells compared to other non-CD45RC⁺ T cells of a mammal. Then, after raising antibodies directed against the CD45RC cell surface marker as above described, the skilled man in the art can easily select those that act on CD45RC⁺ cells, and that can be used to deplete CD45RC^{high} cells via antibody-dependent cell mediated cytotoxicity (ADCC), complement dependent cytotoxicity (CDC), or induction of CD45RC^{high} but not CD45RC^{low/-} cell death (e.g., via apoptosis) after direct binding of the antibody (Picarda et al., 2017. JCI Insight. 2(3):e90088).

“CDR” or “Complementarity Determining Region”

As used herein, the term “CDR” or “complementarity determining region” means the non-contiguous antigen combining sites found within the variable region of both heavy and light chain polypeptides. CDRs were identified according to the rules of Table 1, as deduced from Kabat et al., 1991. Sequences of proteins of immunological interest (5^{th} ed.). Bethesda, Md.: U.S. Dep. of Health and Human Services; and Chothia and Lesk, 1987. J Mol Biol. 196(4):901-17:

TABLE 1
Heavy chain variable region (HCVR or V_{H})

| | V_{H}-CDR1 | V_{H}-CDR2 | V_{H}-CDR3 |
|---|---|---|---|
| Start | Approx. at residue 26 | Always 15 residues after | Always 33 residues after |
| | (always 4 after a Cys) | the end of V_{H}-CDR1 | end of V_{H}-CDR2 |
| | according to | according to Kabat/AbM′s | Always 2 residues after a |
| | Chothia/AbM′s definition | definition | Cys |
| | Kabat′s definition starts 5 | | |
| | residues later | | |
| Residue | Always Cys-Xaa-Xaa-Xaa, | Typically, Leu-Glu-Trp-Ile- | Always Cys-Xaa-Xaa, with |
| before | with Xaa being any amino | Gly, but a number of | Xaa being any amino acid |
| | acid according to | variations | Typically, Cys-Ala-Arg |
| | Chothia/AbM′s definition | | |
| Residue | Always Trp | Lys/Arg- | Always Trp-Gly-Xaa-Gly, |
| after | Typically, Trp-Val, but | Leu/Ile/Val/Phe/Thr/Ala- | with Xaa being any amino |
| | also, Trp-Ile or Trp-Ala | Thr/Ser/Ile/Ala | acid |
| Length | 10 to 12 residues according | 16 to 19 residues according | 3 to 25 residues |
| | to AbM′s definition | to Kabat′s definition | |
| | Chothia′s definition | AbM′s definition ends 7 | |
| | excludes the last 4 residues | residues earlier | |
| | 5 to 7 residues according to | | |
| | Kabat′s definition | | |

Light chain variable region (LCVR or V_{L})

| | V_{L}-CDR1 | V_{L}-CDR2 | V_{L}-CDR3 |
|---|---|---|---|
| Start | Approx. at residue 24 | Always 16 residues after | Always 33 residues after |
| | | the end of V_{L}-CDR1 | end of V_{L}-CDR2 (except |
| | | | NEW (PDB ID: 7FAB) |
| | | | which has the deletion at |
| | | | the end of CDR-L2*) |
| Residue | Always Cys | Generally, Ile-Tyr, but also, | Always Cys |
| before | | Val-Tyr, Ile-Lys or Ile-Phe | |
| Residue | Always Trp | | Always Phe-Gly-Xaa-Gly, |
| after | Typically, Trp-Tyr-Gln, but | | with Xaa being any amino |
| | also, Trp-Leu-Gln, Trp- | | acid |
| | Phe-Gln or Trp-Tyr-Leu | | |
| Length | 10 to 17 residues | Always 7 residues (except | 7 to 11 residues |
| | | NEW (PDB ID: 7FAB) | |
| | | which has a deletion in this | |
| | | region*) | |

*Saul & Poljak, 1992. Proteins. 14(3):363-71

“Chimeric Antibody”

By “chimeric” antibody, it is meant an antibody which contains a natural variable region (light chain and heavy chain) derived from an antibody of a given species associated with constant regions of light chain and heavy chain of an antibody of a species heterologous to said given species. Advantageously, if the monoclonal antibody according to the invention is a chimeric monoclonal antibody, the latter comprises human constant regions. Starting from a non-human antibody, a chimeric antibody may be prepared by using genetic recombinant techniques well known to one skilled in the art. For example, the chimeric antibody may be produced by cloning for the heavy chain and the light chain a recombinant DNA including a promoter and a sequence coding for the variable region of the non-human antibody, and a sequence coding for the constant region of a human antibody. As for the methods for preparing chimeric antibodies, reference may be made, e.g., to Verhoeyn et al. (1988. Science. 239(4847):1534-6).

“Complement Dependent Cytotoxicity” or “CDC”

The terms “complement dependent cytotoxicity” or “CDC” refer to the lysis of a target cell in the presence of complement. Activation of the classical complement pathway is initiated by the binding of the first component of the complement system to antibodies which are bound to their cognate antigen. To assess complement activation, a CDC assay such as, e.g., the one described in Gazzano-Santoro et al., 1997. J Immunol Methods. 202(2):163-71, may be performed.

“Deficient Gene”

As used herein, the term “deficient gene” relates to a gene that is mutated, absent or non-functional. As a consequence, the protein encoded by said deficient gene is missing, or is present in a tiny, non-effective amount, or is under a mutated, inactive form.

“Epitope”

As used herein, the term “epitope” refers to a specific arrangement of amino acids located on a protein or proteins to which an antibody or binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Fragment Crystallizable Region” or “Fc”

As used herein, the term “Fragment crystallizable region” or “Fc” or “Fc region” encompass the polypeptides comprising the constant region of an antibody, excluding the first constant region immunoglobulin domain. Thus, Fc refers to the last two constant region immunoglobulin domains of IgA, IgD and IgG, and to the last three constant region immunoglobulin domains of IgE and IgM and the flexible hinge N-terminal to these domains. For IgA and IgM, Fc may include the J chain. For IgG, Fc comprises immunoglobulin domains Cγ2 and Cγ3 and the hinge between Cγ1 and Cγ2.

Although the boundaries of the Fc region may vary, the human IgG heavy chain Fc region is usually defined to comprise residues C226 or P230 to its carboxyl-terminus, wherein the numbering is according to the EU index as set forth in Kabat et al., 1991 (uences of proteins of immunological interest 5^{th} ed.). Bethesda, Md.: U.S. Dep. of Health and Human Services). The EU index as set forth in Kabat refers to the residue numbering of the human IgG1 EU antibody as described in Kabat et al., 1991 (supra). Fc may refer to this region in isolation, or this region in the context of an antibody, antibody fragment, or Fc fusion protein. The Fc fragment naturally consists of the constant region of the heavy chain excluding domain C_{H}1, i.e., of the lower boundary region and of the constant domains C_{H}2 and C_{H}3 or C_{H}2 to C_{H}4 (depending on the isotype).

In the sense of the invention, the Fc fragment of an antibody may be natural or may have been modified in various ways, provided that it comprises a functional domain for binding to FcR receptors (FcγR receptors for IgGs), and preferably a functional domain for binding to receptor FcRn. The modifications may include the deletion of certain portions of the Fc fragment, provided that the latter contains a functional domain for binding to receptors FcR (receptors FcγR for IgGs), and preferably a functional domain for binding to receptor FcRn. The modifications of the antibody or the Fc fragment of an antibody may also include various substitutions of amino acids able to affect the biological properties of the antibody, provided that the latter contains a functional domain for binding to receptors FcR, and preferably a functional domain for binding to receptor FcRn.

In particular, when the antibody is an IgG, it may comprise mutations intended to enhance the binding to receptor FcγRIII (CD16), as described in WO2000042072, WO2004029207, WO2004063351 or WO2004074455. Mutations permitting to enhance the binding to receptor FcRn, and therefore the half-life in vivo of the antibody, may also be present, as described, for example, in WO2000042072, WO2002060919, WO2010045193 or WO2010106180. Other mutations, such as those permitting to reduce or increase the binding to the proteins of the complement, and therefore the Complement Dependent Cytotoxicity (CDC) response, may be present or not, such as described, for example, in WO1999051642 or WO2004074455.

“Framework Region” or “FR”

As used herein, the term “framework region” or “FR” includes the amino acid residues that are part of the variable region, but are not part of the CDRs (e.g., using the Kabat/Chothia definition of CDRs). Therefore, a variable region framework is between about 100-120 amino acids in length but includes only those amino acids outside of the CDRs.

For the specific example of a HCVR and for the CDRs as defined by Kabat/Chothia:
FR1 may correspond to the domain of the variable region encompassing amino acids 1-25 according to Chothia/AbM's definition, or 5 residues later according to Kabat's definition;
FR2 may correspond to the domain of the variable region encompassing amino acids 36-49;
FR3 may correspond to the domain of the variable region encompassing amino acids 67-98; and
FR4 may correspond to the domain of the variable region from amino acids 104-110 to the end of the variable region.

The framework regions for the light chain are similarly separated by each of the LCVR's CDRs. In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainders of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding site formed by the positioned CDRs defines a surface complementary to the epitope on the immunoreactive antigen. This complementary surface promotes the non-covalent binding of the antibody to the immunoreactive antigen epitope. The position of CDRs can be readily identified by one of ordinary skill in the art.

“Heavy Chain Region”

As used herein, the term “heavy chain region” includes amino acid sequences derived from the constant domains of an immunoglobulin heavy chain. A protein comprising a heavy chain region comprises at least one of a C_{H}1 domain, a hinge (e.g., upper, middle, and/or lower hinge region) domain, a C_{H}2 domain, a C_{H}3 domain, or a variant or fragment thereof. In an embodiment, the antibody or binding fragment thereof according to the present invention may comprise the Fc region of an immunoglobulin heavy chain (e.g., a hinge portion, a C_{H}2 domain, and a C_{H}3 domain). In another embodiment, the antibody or binding fragment thereof according to the present invention lacks at least a region of a constant domain (e.g., all or part of a C_{H}2 domain). In certain embodiments, at least one, and preferably all, of the constant domains are derived from a human immunoglobulin heavy chain. For example, in one preferred embodiment, the heavy chain region comprises a fully human hinge domain In other preferred embodiments, the heavy chain region comprising a fully human Fc region (e.g., hinge, C_{H}2 and C_{H}3 domain sequences from a human immunoglobulin). In certain embodiments, the constituent constant domains of the heavy chain region are from different immunoglobulin molecules. For example, a heavy chain region of a protein may comprise a C_{H}2 domain derived from an IgG1 molecule and a hinge region derived from an IgG3 or IgG4 molecule. In other embodiments, the constant domains are chimeric domains comprising regions of different immunoglobulin molecules. For example, a hinge may comprise a first region from an IgG1 molecule and a second region from an IgG3 or IgG4 molecule. As set forth above, it will be understood by one of ordinary skill in the art that the constant domains of the heavy chain region may be modified such that they vary in amino acid sequence from the naturally occurring (wild-type) immunoglobulin molecule. That is, the antibody or binding fragment thereof according to the present invention may comprise alterations or modifications to one or more of the heavy chain constant domains (C_{H}1, hinge, C_{H}2 or C_{H}3) and/or to the light chain constant domain (C_{L}). Exemplary modifications include additions, deletions or substitutions of one or more amino acids in one or more domains.

“Hinge Region”

As used herein, the term “hinge region” includes the region of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., 1998. J Immunol. 161(8):4083-90).

“Humanized Antibody or Binding Fragment Thereof”

A “humanized antibody or binding fragment thereof”, as used herein, refers to a chimeric antibody or binding fragment thereof which contains minimal sequence derived from a non-human immunoglobulin. It includes antibodies made by a non-human cell having variable and constant regions which have been altered to more closely resemble antibodies that would be made by a human cell, e.g., by altering the non-human antibody amino acid sequence to incorporate amino acids found in human germline immunoglobulin sequences. Humanized antibodies or binding fragment thereof according to the present invention may include amino acid residues not encoded by human germline immunoglobulin sequences (e.g., mutations introduced by random or site-specific mutagenesis in vitro or by somatic mutation in vivo), for example in the CDRs. The term “humanized antibody or binding fragment thereof” also includes antibodies and binding fragment thereof in which CDR sequences derived from the germline of another mammalian species, such as a mouse, have been grafted onto human framework sequences. In other words, the term “humanized antibody or binding fragment thereof” refers to an antibody or binding fragment thereof in which the CDRs of a recipient human antibody are replaced by CDRs from a donor non-human antibody, e.g., a mouse antibody. Humanized antibodies or binding fragments thereof may also comprise residues of donor origin in the framework sequences. The humanized antibody or binding fragment thereof can also comprise at least a portion of a human immunoglobulin constant region. Humanized antibodies and binding fragments thereof may also comprise residues which are found neither in the recipient antibody, nor in the imported CDR or FR sequences. Humanization can be performed using methods known in the art (e.g., Jones et al., 1986. Nature. 321(6069):522-5; Riechmann et al., 1988. Nature. 332(6162):323-7; Verhoeyen et al., 1988. Science. 239(4847):1534-6; Presta, 1992. Curr Opin Biotechnol. 3(4):394-8; Patent US4,816,567), including techniques such as “superhumanizing” antibodies (e.g., Tan et al., 2002. J Immunol. 169(2):1119-25) and “resurfacing” (e.g., Staelens et al., 2006. Mol Immunol. 43(8):1243-57; Roguska et al., 1994. Proc Natl Acad Sci USA. 91(3):969-73).

Methods for humanizing the antibody or binding fragment thereof according to the present invention are well-known in the art, and will be further detailed in the Example section below. The choice of human variable domains, both light and heavy, to be used in making the humanized antibody or binding fragment thereof is very important to reduce antigenicity. According to the so-called “best-fit” method, the sequence of the variable domain of an antibody or binding fragment thereof according to the present invention is screened against the entire library of known human variable-domain sequences. The human sequence that is closest to the mouse sequence is then accepted as the human framework (FR) for the humanized antibody (Sims et al., 1993. J Immunol. 151(4):2296-308; Chothia & Lesk, 1987. J Mol Biol. 196(4):901-17).

Another method for humanizing the antibody or binding fragment thereof according to the present invention uses a particular FR from the consensus sequence of all human antibodies of a particular subgroup of light or heavy chains. The same framework can be used for several different humanized antibodies (Carter et al., 1992. Proc Natl Acad Sci USA. 89(10):4285-9; Presta et al., 1993. J Immunol. 151(5):2623-32). It is further important that antibodies be humanized with retention of high affinity for hCD45RC and other favorable biological properties. To achieve this goal, according to a preferred method, humanized antibodies and binding fragments thereof are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its epitope. In this way, CDR residues can be selected and combined from the consensus and import sequences so that the desired antibody characteristic, such as an increased affinity for hCD45RC, is achieved. In general, the CDR residues are directly and most substantially involved in influencing antigen binding.

Another method for humanizing the antibody or binding fragment thereof according to the present invention is to use a transgenic or transchromosomic animal carrying parts of the human immune system for immunization. As a host, these animals have had their immunoglobulin genes replaced by functional human immunoglobulin genes. Thus, antibodies produced by these animals or in hybridomas made from the B cells of these animals are already humanized Examples of such transgenic or transchromosomic animal include, without limitation:
the XenoMouse (Abgenix, Fremont, Calif.), described in Patents U.S. Pat. Nos. 5,939,598, 6,075,181, 6,114,598, 6,150,584 and 6,162,963;
the HuMAb Mouse® (Medarex, Inc.), described in Lonberg et al., 1994. Nature. 368(6474):856-859; Lonberg & Huszar, 1995. Int Rev Immunol. 13(1):65-93; Harding & Lonberg, 1995. Ann N Y Acad Sci. 764:536-46; Taylor et al., 1992. Nucleic Acids Res. 20(23):6287-95; Chen et al., 1993. Int Immunol. 5(6):647-56; Tuaillon et al., 1993. Proc Natl Acad Sci USA. 90(8):3720-4; Choi et al., 1993. Nat Genet. 4(2):117-23; Chen et al., 1993. EMBO J. 12(3):821-30; Tuaillon et al., 1994. J Immunol. 152(6):2912-20; Taylor et al., 1994. Int Immunol. 6(4):579-91; Fishwild et al., 1996. Nat Biotechnol. 14(7): 845-51;
the KM Mouse®, described in Patent application WO2002043478;
the TC mice, described in Tomizuka et al., 2000. Proc Natl Acad Sci USA. 97(2):722-7; and
the OmniRat™ (OMT, Inc.), described in Patent application WO2008151081; Geurts et al., 2009. Science. 325(5939):433; Menoret et al., 2010. Eur J Immunol. 40(10):2932-41; Osborn et al., 2013. J Immunol. 190(4):1481-90.

Humanized antibodies and binding fragments thereof may also be produced according to various other techniques, such as by using, for immunization, other transgenic animals that have been engineered to express a human antibody repertoire (Jakobovitz et al., 1993. Nature. 362(6417):255-8), or by selection of antibody repertoires using phage display methods. Such techniques are known to the skilled person and can be implemented starting from monoclonal antibodies or binding fragments thereof as disclosed in the present application.

In some embodiments, the antibody or binding fragment thereof according to the present invention comprising HCVR and LCVR (or CDRs thereof) may comprise a first constant domain (C_{H}1 and/or C_{L}), the amino acid sequence of which is fully or substantially human. In some embodiment, especially when the antibody or binding fragment thereof according to the present invention is intended for human therapeutic uses, it is typical for the entire constant region, or at least a part thereof, to have a fully or substantially human amino acid sequence. Therefore, one or more of, or any combination of, the C_{H}1 domain, hinge region, C_{H}2 domain, C_{H}3 domain and C_{L} domain (and C_{H}4 domain if present) may be fully or substantially human with respect to its amino acid sequence. Advantageously, the C_{H}1 domain, hinge region, C_{H}2 domain, C_{H}3 domain and C_{L} domain (and C_{H}4 domain if present) may all have a fully or substantially human amino acid sequence.

The term “substantially human”, in the context of the constant region of a humanized or chimeric antibody or binding fragment thereof, refers to an amino acid sequence identity of at least 70%, preferably at least 75%, 80%, 85%, 90%, 95%, 96%, 97%, 98%, 99% or more with a human constant region.

The term “human amino acid sequence”, in this context, refers to an amino acid sequence which is encoded by a human immunoglobulin gene, which includes germline, rearranged and somatically mutated genes. The present invention also contemplates proteins comprising constant domains of “human” sequence which have been altered, by one or more amino acid additions, deletions or substitutions with respect to the human sequence, excepting those embodiments where the presence of a “fully human hinge region” is expressly required. The presence of a “fully human hinge region” in the antibody or binding fragment thereof. according to the present invention may be beneficial both to minimize immunogenicity and to optimize stability of the antibody. It is considered that one or more amino acid substitutions, insertions or deletions may be made within the constant region of the heavy and/or the light chain, particularly within the Fc region Amino acid substitutions may result in replacement of the substituted amino acid with a different naturally occurring amino acid, or with a non-natural or modified amino acid. Other structural modifications are also permitted, such as for example changes in glycosylation pattern (e.g., by addition or deletion of N- or O-linked glycosylation sites). Depending on the intended use of the antibody or binding fragment thereof, it may be desirable to modify the antibody or binding fragment thereof according to the present invention with respect to its binding properties to Fc receptors, for example to modulate effector function. For example, cysteine residue(s) may be introduced in the Fc region, thereby allowing interchain disulfide bond formation in this region. The homodimeric antibody thus generated may have improved effector function (Caron et al., 1992. J Exp Med. 176(4):1191-5; Shopes, 1992. J Immunol. 148(9):2918-22).

“Hypervariable Loop”

The term “hypervariable loop” is not strictly synonymous to complementarity determining region (CDR), since the hypervariable loops (HVs) are defined on the basis of structure, whereas CDRs are defined based on sequence variability (Kabat et al., 1991. Sequences of proteins of immunological interest (5^{th} ed.). Bethesda, Md.: U.S. Dep. of Health and Human Services) and the limits of the HVs and the CDRs may be different in some V_{H} and V_{L} domains. The CDRs of the V_{L} and V_{H} domains can typically be defined by the Kabat/Chothia definition as already explained hereinabove.

“Identity” or “Identical”

As used herein, the term “identity” or “identical”, when used in a relationship between the sequences of two or more amino acid sequences, or of two or more nucleic acid sequences, refers to the degree of sequence relatedness between amino acid sequences or nucleic acid sequences, as determined by the number of matches between strings of two or more amino acid residues or nucleic acid residues. “Identity” measures the percent of identical matches between the smaller of two or more sequences with gap alignments (if any) addressed by a particular mathematical model or computer program (i.e., “algorithms”) Identity of related amino acid sequences or nucleic acid sequences can be readily calculated by known methods. Such methods include, but are not limited to, those described in Lesk A. M. (1988). Computational molecular biology: Sources and methods for sequence analysis. New York, N.Y.: Oxford University Press; Smith D. W. (1993). Biocomputing: Informatics and genome projects. San Diego, Calif.: Academic Press; Griffin A. M. & Griffin H. G. (1994). Computer analysis of sequence data, Part 1. Totowa, N.J.: Humana Press; von Heijne G. (1987). Sequence analysis in molecular biology: treasure trove or trivial pursuit. San Diego, Calif.: Academic press; Gribskov M. R. & Devereux J. (1991). Sequence analysis primer. New York, N.Y.: Stockton Press; Carillo et al., 1988. SIAM J Appl Math. 48(5):1073-82.

Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, Wis.; Devereux et al., 1984. Nucleic Acids Res. 12(1 Pt 1):387-95), BLASTP, BLASTN, and FASTA (Altschul et al., 1990. J Mol Biol. 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Immune Tolerance”

The term “immune tolerance”, as used herein, relates to a state of unresponsiveness of the immune system to specific substances or tissues that have the capacity to elicit an immune response while preserving immune response against other substances or tissues.

“Immune Response”

The term “immune response”, as used herein, includes T cell-mediated and/or B cell-mediated immune responses. Exemplary immune responses include, but are not limited to, T cell responses (e.g., cytokine production and cellular cytotoxicity), but also immune responses that are indirectly effected by T cell activation (e.g., macrophages) Immune cells involved in the immune response include lymphocytes (such as B cells and T cells, including CD4⁺, CD8⁺, Tₕ1 and Tₕ2 cells), antigen presenting cells (e.g., professional antigen presenting cells such as dendritic cells), natural killer cells, myeloid cells (such as macrophages, eosinophils, mast cells, basophils, and granulocytes).

“Immunospecific”, “Specific for” or “Specifically Bind”

As used herein, an antibody or binding fragment thereof is said to be “immunospecific”, “specific for” or to “specifically bind” an antigen if it reacts at a detectable level with said antigen (e.g., SEQ ID NO: 1), preferably with an affinity constant (K_{A}) of greater than or equal to about 10⁶M⁻¹, preferably greater than or equal to about 10⁷M⁻¹, 10⁸ M⁻¹, 5×10⁸ M⁻¹, 10⁹ M⁻¹, 5×10⁹ M⁻¹ or more.

Affinity of an antibody or binding fragment thereof for its cognate antigen is also commonly expressed as an equilibrium dissociation constant (K_{D}). an antibody or binding fragment thereof is said to be “immunospecific”, “specific for” or to “specifically bind” an antigen if it reacts at a detectable level with said antigen (e.g., SEQ ID NO: 1), preferably with a K_{D} of less than or equal to 10⁻⁶ M, preferably less than or equal to 10⁻⁷ M, 5×10⁻⁸ M, 10⁻⁸ M, 5×10⁻⁹ M, 10⁻⁹ M or less.

Affinities of antibodies or binding fragment thereof can be readily determined using conventional techniques, for example, those described by Scatchard, 1949. Ann NY Acad Sci.

51:660-672. Binding properties of an antibody or binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR, e.g., using BlAcore®).

“Monoclonal Antibody”

As used herein, the term “monoclonal antibody” refers to an antibody obtained from a population of substantially homogeneous antibodies, i.e., the individual antibodies comprised in the population are identical except for possible naturally occurring mutations that may be present in minor amounts. For example, individual antibodies may vary as regards their post—translational modifications, and notably as regards their glycosylation structures or their isoelectric point, but have all been encoded by the same heavy and light chain sequences and therefore have, before any post-translational modification, the same protein sequence. Certain differences in protein sequences, related to post-translational modifications (such as for example the cleavage of the C-terminal lysine of the heavy chain, deamidation of asparagine residues and/or isomerization of aspartate residues), may nevertheless exist between individual antibodies present in a monoclonal antibody composition.

Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies or binding fragment thereof according to the present invention may be prepared by the hybridoma methodology first described by Kohler et al., 1975. Nature. 256(5517):495-7, or may be made using recombinant DNA methods in bacterial, eukaryotic animal or plant cells (Patent U.S. Pat. No. 4,816,567). The “monoclonal antibodies” may also be isolated from phage antibody libraries using the techniques described in Clackson et al., 1991. Nature. 352(6336):624-8 and Marks et al., 1991. J Mol Biol. 222(3):581-97, for example.

“Monogenic Disease”

The term “monogenic disease”, also called “single-gene disorder”, refers to a genetic disorder in which modifications of a single gene is associated with a disorder, disease, or condition in a subject. Though relatively rare, monogenic diseases affect millions of people worldwide. Scientists currently estimate that over 10,000 human diseases are known to be monogenic. Pure genetic diseases are caused by a single error in a single gene in the human DNA. The nature of disease depends on the functions performed by the modified gene. The single-gene or monogenic diseases can be classified into six categories: autosomal dominant, autosomal recessive, X-linked dominant, X-linked recessive, Y-linked and mitochondrial, depending on the mutated gene itself and its occurrence on one or two alleles.

Some examples of monogenic diseases include, but are not limited to, 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis; anauxetic dysplasia; androgen insentivity syndrome; Antley-Bixler syndrome; APECED; Apert syndrome; aplasia of lacrimal and salivary glands; argininemia; arrhythmogenic right ventricular dysplasia (ARVD2); Arts syndrome; arylsulfatase deficiency type metachromatic leukodystrophy; ataxia telangiectasia; autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL syndrome; carbamyl phosphate synthetase deficiency; cardiofaciocutaneous syndrome; Carney triad; carnitine palmitoyltransferase deficiencies; cartilage-hair hypoplasia; cb1C type of combined methylmalonic aciduria; CD18 deficiency; CD3Z-associated primary T-cell immunodeficiency; CD4OL deficiency; CDAGS syndrome; CDG1a; CDG1b; CDG1m; CDG2c; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile acid synthesis defect type 1; congenital bile acid synthesis defect type 2; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosis; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT-1 deficiency; glycogen storage disease type lb; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasia; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborn errors of thyroid hormone biosynthesis; infantile visceral myopathy; infantile X-linked spinal muscular atrophy; intrahepatic cholestasis of pregnancy; IPEX syndrome; IRAK4 deficiency; isolated congenital asplenia; Jeune syndrome; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann conical dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease.

“Subject”

As used herein, the term “subject” refers to a mammal, preferably a human In one embodiment, a subject may be a “patient”, i.e., a warm-blooded animal, more preferably a human, who/which is awaiting the receipt of, or is receiving medical care or was/is/will be the object of a medical procedure, or is monitored for the development of a disease. The term “mammal” refers here to any mammal, including humans, domestic and farm animals, and zoo, sports, or pet animals, such as dogs, cats, cattle, horses, sheep, pigs, goats, rabbits, etc. Preferably, the mammal is a primate, more preferably a human

“Therapeutically Effective Amount”

As used herein, the expression “therapeutically effective amount” means the level, amount or concentration of agent (e.g., an anti-CD45RC antibody) that is aimed at, without causing significant negative or adverse side effects to the subject, (1) delaying or preventing the onset of monogenic diseases; (2) slowing down or stopping the progression, aggravation, or deterioration of one or more symptoms of monogenic diseases; (3) bringing about ameliorations of the symptoms of monogenic diseases; (4) reducing the severity or incidence of monogenic diseases; or (5) curing monogenic diseases. A therapeutically effective amount may be administered prior to the onset of the monogenic disease, for a prophylactic or preventive action. Alternatively or additionally, the therapeutically effective amount may be administered after initiation of the monogenic disease, for a therapeutic action.

“Treating” or “Treatment” or “Alleviation”

As used herein, the terms “treating” or “treatment” or “alleviation” refer to both therapeutic and prophylactic (or preventative) measures; wherein the object is to slow down (or lessen) the targeted pathologic condition or disorder. Those in need of treatment include those already with the monogenic disease as well those suspected to have the monogenic disease. A subject is successfully “treated” for the targeted monogenic disease if, after receiving a therapeutically effective amount of the agent (e.g., an anti-CD45RC antibody), said subject shows observable and/or measurable reduction in or absence of one or more of the following: reduction in the number of CD45RC⁺ cells; reduction in the percent of total cells that are CD45RC⁺; relief to some extent (such as reduction, alleviation, lessening or inhibition), of one or more of the symptoms associated with the monogenic disease; reduced morbidity and mortality; and/or improvement in quality of life issues. The above parameters for assessing successful treatment and improvement in the monogenic disease are readily measurable by routine procedures familiar to a physician.

“Variable Region” or “Variable Domain”

As used herein, the term “variable” refers to the fact that certain regions of the variable domains V_{H} and V_{L} differ extensively in sequence among antibodies and are used in the binding and specificity of each particular antibody for its target antigen. However, the variability is not evenly distributed throughout the variable domains of antibodies. It is concentrated in three segments called “hypervariable loops” in each of the V_{L} domain and the V_{H} domain which form part of the antigen binding site.

The first, second and third hypervariable loops of the Vλ light chain domain are referred to herein as L1 (λ), L2 (λ) and L3 (λ) and may be defined as comprising residues 24-33 (L1(λ), consisting of 9, 10 or 11 amino acid residues), 49-53 L2 (λ), consisting of 3 residues) and 90-96 (L3(λ), consisting of 6 residues) in the V_{L} domain (Morea et al., 2000. Methods. 20(3):267-79).

The first, second and third hypervariable loops of the V_{κ} light chain domain are referred to herein as L1(κ), L2(κ) and L3(κ) and may be defined as comprising residues 25-33 (L1(κ), consisting of 6, 7, 8, 11, 12 or 13 residues), 49-53 (L2(κ), consisting of 3 residues) and 90-97 (L3(κ), consisting of 6 residues) in the V_{L} domain (Morea et al., 2000. Methods. 20(3):267-79).

The first, second and third hypervariable loops of the V_{H} domain are referred to herein as H1, H2 and H3 and may be defined as comprising residues 25-33 (H1, consisting of 7, 8 or 9 residues), 52-56 (H2, consisting of 3 or 4 residues) and 91-105 (H3, highly variable in length) in the V_{H} domain (Morea et al., 2000. Methods. 20(3):267-79).

Unless otherwise indicated, the terms L1, L2 and L3 respectively refer to the first, second and third hypervariable loops of a V_{L} domain, and encompass hypervariable loops obtained from both Vκ and Vλ isotypes. The terms H1, H2 and H3 respectively refer to the first, second and third hypervariable loops of the V_{H} domain, and encompass hypervariable loops obtained from any of the known heavy chain isotypes, including gamma (γ), mu (μ), alpha (α), delta (δ) or epsilon (ε). The hypervariable loops L1, L2, L3, H1, H2 and H3 may each comprise part of a “complementarity determining region” or “CDR”, as defined hereinabove.

### DETAILED DESCRIPTION

The present invention relates to anti-CD45RC antibodies or binding fragments thereof, for use in the prevention and/or treatment of monogenic diseases.

In one embodiment, anti-CD45RC antibody or binding fragment thereof for use according to the invention is isolated. The term “isolated”, as used herein in reference to an antibody or binding fragment thereof, means that said antibody or binding fragment thereof is substantially free of other antibodies or binding fragments having different antigenic specificities (e.g., an isolated antibody that specifically binds CD45RC is substantially free of antibodies that specifically bind antigens other than CD45RC). An isolated antibody that specifically binds CD45RC from one species may, however, have cross-reactivity to other antigens, such as CD45RC from other species. Moreover, an isolated antibody may be substantially free of other cellular material and/or chemicals, in particular those that would interfere with diagnostic or therapeutic uses of the antibody, including without limitation, enzymes, hormones, and other proteinaceous or non-proteinaceous components.

In one embodiment, the anti-CD45RC antibody or binding fragment thereof for use according to the invention is purified. In one embodiment, the anti-CD45RC antibody or binding fragment thereof for use according to the invention is purified to:
(1) greater than 80%, 85%, 90%, 91%, 92%, 93%, 94%, 95% or more by weight of antibody or binding fragment thereof as determined by the Lowry method, and most preferably more than 96%, 97%, 98% or 99% by weight;
(2) a degree sufficient to obtain at least 15 residues of N-terminal or internal amino acid sequence by use of a spinning cup sequenator; or
(3) homogeneity as shown by SDS-PAGE under reducing or non-reducing conditions and using Coomassie blue or, preferably, silver staining.

In one embodiment, the anti-CD45RC antibody or binding fragment thereof for use according to the present invention binds to CD45RC. In one embodiment, the antibody or binding fragment thereof for use according to the present invention binds to human CD45RC (hCD45RC).

In one embodiment, the anti-CD45RC antibody or binding fragment thereof for use according to the present invention binds to the C determinant encoded by exon 6 of hCD45RC. In one embodiment, the anti-CD45RC antibody or binding fragment thereof for use according to the present invention binds to at least one epitope on the C determinant encoded by exon 6 of hCD45RC.

In one embodiment, the amino acid sequence of the C determinant encoded by exon 6 of hCD45RC comprises or consists of SEQ ID NO: 1. In one embodiment, the nucleic acid sequence of exon 6 encoding the C determinant of hCD45RC comprises or consists of SEQ ID NO: 2.

SEQ ID NO: 1
DVPGERSTASTFPTDPVSPLTTTLSLAHHSSAALPARTSNTTITANTS
SEQ ID NO: 2
GATGTCCCAGGAGAGAGGAGTACAGCCAGCACCTTTCCTACAGACCCA
GTTTCCCCATTGACAACCACCCTCAGCCTTGCACACCACAGCTCTGCT
GCCTTACCTGCACGCACCTCCAACACCACCATCACAGCGAACACCTCA

In one embodiment, the anti-CD45RC antibody or binding fragment thereof for use according to the present invention binds to at least one epitope comprising or consisting of SEQ ID NO: 1 or a fragment thereof.

In one embodiment, the anti-CD45RC antibody or binding fragment thereof for use according to the present invention binds to at least one epitope comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 1 or a fragment thereof.

In one embodiment, the anti-CD45RC antibody or binding fragment thereof for use according to the present invention binds to at least one epitope encoded by a nucleic acid sequence comprising or consisting of SEQ ID NO: 2 or a fragment thereof.

In one embodiment, the anti-CD45RC antibody or binding fragment thereof for use according to the present invention binds to at least one epitope encoded by a nucleic acid sequence comprising or consisting of a sequence sharing at least about 70%, preferably at least 75%, 80%, 85%, 90%, 91%, 92%, 93%, 94%, 95%, 96%, 97%, 98%, 99% or more identity with SEQ ID NO: 2 or a fragment thereof.

In one embodiment, the anti-CD45RC antibody or binding fragment thereof for use according to the present invention binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acids of SEQ ID NO: 1 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 1 or a fragment thereof.

In one embodiment, the anti-CD45RC antibody or binding fragment thereof for use according to the present invention binds to at least one epitope comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 contiguous amino acids of SEQ ID NO: 1 or a fragment thereof; or of a sequence sharing at least about 70%, preferably at least 75%, 80%, 85%, 90%, 91%, 92%, 93%, 94%, 95%, 96%, 97%, 98%, 99% or more identity with SEQ ID NO: 1 or a fragment thereof.

In one embodiment, a fragment of the at least one epitope comprising or consisting of SEQ ID NO: 1 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues.

In one embodiment, a fragment of the at least one epitope comprising or consisting of SEQ ID NO: 1 comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 amino acid residues spread over a span of 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 73, 740, 750, 760, 770, 780, 790, 800, 810, 820, 830, 840, 850, 860, 870, 880, 890, 900 ,910, 920, 930, 940, 950, 960, 970, 980, 990, 1000, 1010, 1020, 1030, 1040, 1050, 1060, 1070, 1080, 1090, 1100 or more contiguous amino acid residues of a sequence comprising or consisting of SEQ ID NO: 1.

In one embodiment, a sequence comprising SEQ ID NO: 1 is SEQ ID NO: 3, corresponding to UniProt Accession P08575-3 (version 3, modified Mar. 28, 2018—Checksum: 6E942E2BF6B17AC5), or a sequence sharing at least about 70%, preferably at least 75%, 80%, 85%, 90%, 91%, 92%, 93%, 94%, 95%, 96%, 97%, 98%, 99% or more identity with SEQ ID NO: 3.

SEQ ID NO: 3
MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDVPG
ERSTASTFPTDPVSPLTTTLSLAHHSSAALPARTSNTTITANTSDAYLNA
SETTTLSPSGSAVISTTTIATTPSKPTCDEKYANITVDYLYNKETKLFTA
KLNVNENVECGNNTCTNNEVHNLTECKNASVSISHNSCTAPDKTLILDVP
PGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFC
RSEAAHQGVITWNPPQRSFHNFTLCYIKETEKDCLNLDKNLIKYDLQNLK
PYTKYVLSLHAYIIAKVQRNGSAAMCHFTTKSAPPSQVWNMTVSMTSDNS
MHVKCRPPRDRNGPHERYHLEVEAGNTLVRNESHKNCDFRVKDLQYSTDY
TFKAYFHNGDYPGEPFILHHSTSYNSKALIAFLAFLIIVTSIALLVVLYK
IYDLHKKRSCNLDEQQELVERDDEKQLMNVEPIHADILLETYKRKIADEG
RLFLAEFQSIPRVFSKFPIKEARKPFNQNKNRYVDILPYDYNRVELSEIN
GDAGSNYINASYIDGFKEPRKYIAAQGPRDETVDDFWRMIWEQKATVIVM
VTRCEEGNRNKCAEYWPSMEEGTRAFGDVVVKINQHKRCPDYIIQKLNIV
NKKEKATGREVTHIQFTSWPDHGVPEDPHLLLKLRRRVNAFSNFFSGPIV
VHCSAGVGRTGTYIGIDAMLEGLEAENKVDVYGYVVKLRRQRCLMVQVEA
QYILIHQALVEYNQFGETEVNLSELHPYLHNMKKRDPPSEPSPLEAEFQR
LPSYRSWRTQHIGNQEENKSKNRNSNVIPYDYNRVPLKHELEMSKESEHD
SDESSDDDSDSEEPSKYINASFIMSYWKPEVMIAAQGPLKETIGDFWQMI
FQRKVKVIVMLTELKHGDQEICAQYWGEGKQTYGDIEVDLKDTDKSSTYT
LRVFELRHSKRKDSRTVYQYQYTNWSVEQLPAEPKELISMIQVVKQKLPQ
KNSSEGNKHHKSTPLLIHCRDGSQQTGIFCALLNLLESAETEEVVDIFQV
VKALRKARPGMVSTFEQYQFLYDVIASTYPAQNGQVKKNNHQEDKIEFDN
EVDKVKQDANCVNPLGAPEKLPEAKEQAEGSEPTSGTEGPEHSVNGPASP
ALNQGS

In one embodiment, the at least one epitope is a conformational epitope. In another embodiment, the at least one epitope is a sequential epitope.

In one embodiment, the anti-CD45RC antibody or binding fragment thereof for use according to the present invention binds to hCD45RC with an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less.

In one embodiment, the anti-CD45RC antibody or binding fragment thereof for use according to the present invention binds to hCD45RC with an association rate (Kₒₙ) of about 1×10⁴ M⁻¹sec⁻¹ or more, preferably of about 5×10⁴ M⁻'sec⁻¹ or more, about 1×10⁵ M⁻'sec⁻¹ or more, about 2.5×10⁵ M⁻'sec⁻¹ or more, about 5×10⁵ M⁻'sec⁻¹ or more.

In one embodiment, the anti-CD45RC antibody or binding fragment thereof for use according to the present invention binds to hCD45RC with a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

In one embodiment, the anti-C′D45RC antibody or binding fragment thereof for use according to the present invention binds to hCD45RC with at least one of, preferably at least two of, more preferably the three of:
an equilibrium dissociation constant (K_{d}) of about 5×10⁻⁷ M or less, preferably of about 2.5×10⁻⁷ M or less, about 1×10⁻⁷ M or less, about 7.5×10⁻⁸ M or less, about 5×10⁻⁸ M or less, about 1×10⁻⁸ M or less;
an association rate (Kₒₙ) of about 1×10⁴ M⁻'sec⁻¹ or more, preferably of about 5×10⁴ M⁻¹sec⁻¹ or more, about 1×10⁵ M⁻'sec⁻¹ or more, about 2.5×10⁵ M⁻'sec⁻¹ or more, about 5×10⁵ M⁻'sec⁻¹ or more; and
a dissociation rate (K_{off}) of about 5×10⁻² sec⁻¹ or less, preferably of about 4×10⁻² sec⁻¹ or less, about 3×10⁻² sec⁻¹ or less, about 2×10⁻² sec⁻¹ or less, about 1.5×10⁻² sec⁻¹ or less.

Methods for determining the affinity (including, for example, determining the K_{d}, k_{off} and kₒₙ) of an antibody or binding fragment thereof for its ligand are well-known in the art, and include, without limitation, surface plasmon resonance (SPR), fluorescence-activated cell sorting (FACS), enzyme-linked immunosorbent assay (ELISA), AlphaLISA and KinExA.

A preferred method is BIAcore®high relies on SPR using immobilized CD45RC to determine the affinity of an antibody or binding fragment thereof. A way of implementing this method will be further illustrated in the Examples section.

In one embodiment, the antibody or binding fragment thereof for use according to the present invention is a polyclonal antibody or binding fragment thereof.

In a preferred embodiment, the antibody or binding fragment thereof for use according to the present invention is a monoclonal antibody or binding fragment thereof.

Antibodies directed against CD45RC can be obtained according to known methods by administering the appropriate antigen or epitope (such as the peptide of SEQ ID NO: 1) to a host animal selected, e.g., from rats, pigs, cows, horses, rabbits, goats, sheep, Camelidae (camel, dromedary, llama) and mice, among others. Various adjuvants known in the art can be used to enhance antibody production. Techniques for production and isolation include but are not limited to the hybridoma technique, the human B-cell hybridoma technique and the EBV-hybridoma technique. Alternatively, techniques described for the production of single chain antibodies (disclosed in, e.g., U.S. Pat. No. 4,946,778) can be adapted to produce single chain antibodies against CD45RC.

In one embodiment, the antibody for use according to the invention is a chimeric antibody or a bispecific antibody or a humanized antibody or a fully human antibody or an antibody fragment or a derivative therefrom. Indeed, this gives the possibility of avoiding immune reactions of the patient against the administered antibodies.

A “derivative” of an antibody means a binding protein formed of a support peptide and at least one CDR of the original antibody, preserving its ability to recognize specifically an antigen, such as CD45RC, in particular SEQ ID NO: 1.

Variable domains are involved in recognition of the antigen, while constant domains are involved in biological, pharmacokinetic and effector properties of the antibody. Unlike variable domains, for which the sequence strongly varies from one antibody to another, constant domains are characterized by an amino acid sequence very close from one antibody to the other, typical of the species and of the isotype, with optionally a few somatic mutations.

In one embodiment, the antibody for use according to the present invention is a multispecific antibody comprising a first antigen binding site directed against CDR45RC and at least one second antigen binding site directed against an effector cell, said effector cell being able to mediate depletion of T CD45RC^{high} cells through direct binding, antibody-dependent cell-mediated cytotoxicity (ADCC), complement dependent cytotoxicity (CDC) and/or antibody dependent phagocytosis.

In said embodiment, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46.

Exemplary formats for the multispecific antibody molecules of the present invention include, but are not limited to
(i) two antibodies cross-linked by chemical hetero-conjugation, one with a specificity to a specific surface molecule of ILC and another with a specificity to a second antigen;
(ii) a single antibody that comprises two different antigen-binding regions;
(iii) a single-chain antibody that comprises two different antigen-binding regions, e.g., two scFvs linked in tandem by an extra peptide linker;
(iv) a dual-variable-domain antibody (DVD-Ig), where each light chain and heavy chain contains two variable domains in tandem through a short peptide linkage;
(v) a chemically-linked bispecific (Fab′)2 fragment;
(vi) a Tandab, which is a fusion of two single chain diabodies resulting in a tetravalent bispecific antibody that has two binding sites for each of the target antigens;
(vii) a flexibody, which is a combination of scFvs with a diabody resulting in a multivalent molecule;
(viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment;
(ix) a so-called Scorpion molecule, comprising, e.g., two scFvs fused to both termini of a human Fab-arm; and
(x) a diabody.

Another exemplary format for bispecific antibodies is IgG-like molecules with complementary C_{H}3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody) (EMD Serono), Biclonic (Merus) and DuoBody (Genmab A/S) technologies.

In some embodiments, a bispecific antibody is obtained or obtainable via a controlled Fab-arm exchange, typically using DuoBody® technology. In vitro methods for producing bispecific antibodies by controlled Fab-arm exchange have been described in WO2008119353 and WO2011131746 (both by Genmab A/S).

In one exemplary method, described in WO2008119353, a bispecific antibody is formed by “Fab-arm” or “half-molecule” exchange (swapping of a heavy chain and attached light chain) between two monospecific antibodies, both comprising IgG4-like C_{H}3 regions, upon incubation under reducing conditions. The resulting product is a bispecific antibody having two Fab arms which may comprise different sequences.

In another exemplary method, described in WO2011131746, bispecific antibodies are prepared by a method comprising the following steps, wherein at least one of the first and second antibodies is a human monoclonal antibody of the present invention:
a) providing a first antibody comprising an Fc region of an immunoglobulin, said Fc region comprising a first C_{H}3 region;
b) providing a second antibody comprising an Fc region of an immunoglobulin, said Fc region comprising a second C_{H}3 region; wherein the sequences of said first and second C_{H}3 regions are different and are such that the heterodimeric interaction between said first and second C_{H}3 regions is stronger than each of the homodimeric interactions of said first and second C_{H}3 regions;
c) incubating said first antibody together with said second antibody under reducing conditions; and
d) obtaining said bispecific antibody, wherein the first antibody is a human monoclonal antibody of the present invention and the second antibody has a different binding specificity, or vice versa.

The reducing conditions may, for example, be provided by adding a reducing agent, e.g., selected from 2-mercaptoethylamine, dithiothreitol and tris(2-carboxyethyl)phosphine. Step d) may further comprise restoring the conditions to become non-reducing or less reducing, for example by removal of a reducing agent, e.g., by desalting. Preferably, the sequences of the first and second C_{H}3 regions are different, comprising only a few, fairly conservative, asymmetrical mutations, such that the heterodimeric interaction between said first and second C_{H}3 regions is stronger than each of the homodimeric interactions of said first and second C_{H}3 regions. More details on these interactions and how they can be achieved are provided in WO2011131746, which is hereby incorporated by reference in its entirety.

The following are exemplary embodiments of combinations of such asymmetrical mutations, optionally wherein one or both Pc-regions are of the IgG1 isotype. In some embodiments, the first Fc region has an amino acid substitution at a position selected from the group consisting of: 366, 368, 370, 399, 405, 407 and 409, and the second Fc region has an amino acid substitution at a position selected from the group consisting of: 366, 368, 370, 399, 405, 407 and 409, and wherein the first and second Fc regions are not substituted in the same positions. In some embodiments, the first Fc region has an amino acid substitution at position 405, and said second Fc region has an amino acid substitution at a position selected from the group consisting of: 366, 368, 370, 399, 407 and 409, optionally 409. In some embodiments, the first Fc region has an amino acid substitution at position 409, and said second Fc region has an amino acid substitution at a position selected from the group consisting of: 366, 368, 370, 399, 405, and 407, optionally 405 or 368. In some embodiments, both the first and second Fc regions are of the IgG1 isotype, with the first Fe region having a Leu at position 405, and the second Fc region having an Arg at position 409. These point mutations, indicated according to Kabat numbering, are well known by the man skilled in the art, since they are disclosed in several patent applications, such as for example in WO2016091891.

The antibodies may be of several isotypes, depending on the nature of their constant region: constant regions γ, α, μ, ε and δ respectively correspond to IgG, IgA, IgM, IgE and IgD immunoglobulins.

In one embodiment, the monoclonal antibody for use according to the present invention is an IgG. In particular, IgG isotype shows an ability to generate ADCC and/or CDC activity in the largest number of individuals (humans). y constant regions comprise several sub-types: γ1, γ2, γ3, these three types of constant regions having the particularity of binding the human complement, and γ4, thereby generating sub-isotypes IgG1, IgG2, IgG3, and IgG4. In one embodiment, the monoclonal antibody according to the invention is of an isotype IgG1 or IgG3, preferably IgG1. The monoclonal antibody may be produced by a cell clone, a non-human transgenic animal or a transgenic plant, by technologies well known to one skilled in the art.

In an alternative embodiment, the monoclonal antibody for use according to the present invention does not comprise a Fc fragment. In particular, antibodies lacking or deprived of a Fc fragment do not generate ADCC or CDC activity. Such antibody is particularly suitable when its use is not ADCC- and/or CDC-dependent.

Suitable examples of anti-CD45RC antibody have been described in WO2016016442, the content of which is specifically incorporated herein by reference in its entirety.

Other suitable examples of monoclonal antibodies directed against CD45RC are well-known from the one skilled in the art and include commercialized antibodies. Examples of such antibodies include, without limitation, the mouse monoclonal antibodies OX-22 (anti-rat CD45RC), OX-32 (anti-rat CD45RC), 3H1437 (anti-mouse/rat/human CD45RC), MT2. (anti-human CD45RC), and RPI/12 (anti-human CD45RC) or derivatives thereof, as well as rat monoclonal antibodies DNL 1.9 (anti-mouse CD45RC) and C455.1F (anti-mouse CD45RC). Such monoclonal antibodies are well-known from the one skilled in the art and are commercialized by several companies (Spickett et al., 1983. J Exp Med. 158(3):795-810).

In a particular embodiment, the anti-CDR45RC antibody for use according to the present invention is a monoclonal antibody OX-22, OX-32, 3H1437, MT2, RP1/12, DNL 1.9, C455.1F or a derivative thereof. Such monoclonal antibodies are well-known from the one skilled in the art and are commercialized by several companies.

The expression “a derivative thereof”, with reference to a monoclonal antibody, refers to an anti-CD45RC antibody which specifically binds to CD45RC, preferably to human CD45RC, and which comprises the 6 CDRs of said monoclonal antibody. In one embodiment, the “derivative thereof” is an antibody which comprises the VL chain and the VH chain of said monoclonal antibody. In another embodiment, the “derivative thereof” is a chimeric antibody or humanized antibody, which comprises the variable domains of said monoclonal antibody.

In one embodiment, the antibody according to the present invention can be modified to enhance antibody-dependent cell-mediated cytotoxicity (ADCC), complement dependent cytotoxicity (CDC) and/or antibody-dependent phagocytosis. Such modifications are well-known in the art.

For example, antibodies comprising a low fucose content are known to enhance ADCC response via the FcγRIII receptor (International patent publication WO2014140322). Thus, the antibody according to the present invention may comprise a low fucose content.

The term “fucose content”, as used herein, refers to the percentage of fucosylated forms within the N-glycans attached to the N297 residue of the Fc fragment of each heavy chain of each antibody.

The term “low fucose content”, as used herein, refers to a fucose content of less than, or equal to, 65%. Advantageously, the fucose content is less than or equal to 65%, preferably less than or equal to 60%, 55% or 50%, or even less than or equal to 45%, 40%, 35%, 30%, 25% or 20%.

However, it is not necessary that the fucose content be zero, and it may for example be greater than or equal to 5%, 35 10%, 15% or 20%.

In one embodiment, the antibody according to the present invention may further comprises different types of glycosylation (N-glycans of the oligomannose or biantennary complex type, with a variable proportion of bisecting N-acetylglucosamine (GlcNAc) residues or galactose residues in the case of N-glycans of the biantennary complex type), provided that they have a low fucose content (International patent publication WO2007048077). For example, antibodies having slightly fucosylated N-glycans can be obtained as described in European patent publication 1176195 or in International patent publications WO2001077181 or WO2012041768.

The N-glycans of the oligomannose type have reduced half-life in vivo as compared to N-glycans of the biantennary complex type. Consequently, advantageously, the antibodies according to the present invention have on their N-glycosylation sites of the Fc fragment glycan structures of the biantennary complex type, with a low fucose content, as defined above.

In some embodiments, the antibody according to the present invention is conjugated to a therapeutic moiety, i.e., a drug. In one embodiment, the therapeutic moiety is selected from a cytotoxin, a chemotherapeutic agent, a cytokine, an immunosuppressant, an immune stimulator, a lytic peptide and a radioisotope. Such conjugates are referred to herein as an “antibodydrug conjugates” or “ADCs”.

In some embodiments, the antibody of the present invention is conjugated to a cytotoxic moiety. In one embodiment, the cytotoxic moiety is selected from taxol; cytochalasin B; gramicidin D; ethidium bromide; emetine; mitomycin; etoposide; tenoposide; vincristine; vinblastine; colchicin; doxorubicin; daunorubicin; dihydroxyanthracin dione; tubulin-inhibitors (such as, e.g., maytansine or an analog or derivative thereof); antimitotic agents (such as, e.g., monomethyl auristatin E or F or an analog or derivative thereof); dolastatin or an analogue thereof; irinotecan or an analogue thereof; mitoxantrone; mithramycin; actinomycin D; 1-dehydrotestosterone; glucocorticoids; procaine; tetracaine; lidocaine; propranolol; puromycin; calicheamicin or an analog or derivative thereof; antimetabolites (such as, e.g., methotrexate, mercaptopurine, 6-thioguanine, cytarabine, fludarabin, 5-fluorouracil, decarbazine, hydroxyurea, asparaginase, gemcitabine, or cladribine); an alkylating agent (such as, e.g., mechlorethamine, thioepa, chlorambucil, melphalan, carmustine, lomustine, cyclophosphamide, busulfan, dibromomannitol, streptozotocin, dacarbazine, procarbazine or mitomycin C); platinum derivatives (such as, e.g., cisplatin or carboplatin); duocarmycin A, duocarmycin SA, rachelmycin, or an analog or derivative thereof; antibiotics (such as, e.g., dactinomycin, bleomycin, daunorubicin, doxorubicin, idarubicin, mithramycin, mitomycin, mitoxantrone, plicamycin or anthramycin); pyrrolo [2,1-c] [1,4]-benzodiazepines; diphtheria toxin and related molecules (such as, e.g., diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alphasarcin, Aleurites fordii proteins, dianthin proteins, Phytolacca americana proteins such as PAPI, PAPII, and PAP-S, momordica charantia inhibitor, curcin, crotin, sapaonaria officinalis inhibitor, gelonin, mitogellin, restrictocin, phenomycin, and enomycin toxins); ribonucleases; DNase I; Staphylococcal enterotoxin A; pokeweed antiviral protein; diphtherin toxin; and Pseudomonas endotoxin.

In some embodiments, the antibody according to the present invention is conjugated to an auristatin or a peptide analog, derivative or prodrug thereof. Auristatins have been shown to interfere with microtubule dynamics, GTP hydrolysis and nuclear and cellular division (Woyke et al., 2001. Antimicrob Agents Chemother. 46(12):3802-8) and have anti-cancer (U.S. Pat. No. 5,663,149) and antifungal activity (Pettit et al., 1998. Antimicrob Agents Chemother. 42(11):2961-5). For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to antibodies, are described in, e.g., U.S. Pat. Nos. 5,635,483, 5,780,588 and 6,214,345 and in International patent publications WO2002088172, WO2004010957, WO2005081711, WO2005084390, WO2006132670, WO2003026577, WO200700860, WO2007011968 and WO2005082023.

In some embodiments, the antibody according to the present invention is conjugated to pyrrolo[2,1-c][1,4]-benzodiazepine (PDB) or an analog, derivative or prodrug thereof. Suitable PDBs and PDB derivatives, and related technologies are described in the art.

In some embodiments, the antibody according to the present invention is conjugated to a cytotoxic moiety selected from anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody according to the present invention is conjugated to an anthracycline or an analog, derivative or prodrug thereof.

In some embodiments, the antibody according to the present invention is conjugated to maytansine or an analog, derivative or prodrug thereof.

In some embodiments, the antibody according to the present invention is conjugated to calicheamicin or an analog, derivative or prodrug thereof.

In some embodiments, the antibody according to the present invention is conjugated to duocarmycin or an analog, derivative or prodrug thereof.

In some embodiments, the antibody according to the present invention is conjugated to rachelmycin (CC-1065) or an analog, derivative or prodrug thereof.

In some embodiments, the antibody according to the present invention is conjugated to dolastatin or an analog, derivative or prodrug thereof.

In some embodiments, the antibody according to the present invention is conjugated to monomethyl auristatin E or an analog, derivative or prodrug thereof.

In some embodiments, the antibody according to the present invention is conjugated to monomethyl auristatin F or an analog, derivative or prodrug thereof.

In some embodiments, the antibody according to the present invention is conjugated to irinotecan or an analog, derivative or prodrug thereof.

Techniques for conjugating molecule to antibodies are well-known in the art (see, e.g., Arnon, 1985. In Reisfeld & Sell, Monoclonal antibodies and cancer therapy (Vol. 27, UCLA symposia on molecular and cellular biology, pp. 243-256). New York, N.Y.: Alan R. Liss; and PCT publication WO1989012624). Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate. Junutula et al. (2008. J Immunol Methods. 332(1-2):41-52) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established. In particular the one skilled in the art can also envisage Fc-containing polypeptide engineered with an acyl donor glutamine-containing tag (e.g., Gin-containing peptide tags or Q-tags) or an endogenous glutamine that are made reactive by polypeptide engineering (e.g., via amino acid deletion, insertion, jsubstitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site-specifically conjugated to the Fc-containing polypeptide through the acyl donor glutamine-containing tag or the accessible/exposed/reactive endogenous glutamine (see, e.g., WO2012059882).

In one embodiment, the anti-CD45RC antibody for use according to the invention is used to prevent and/or treat monogenic diseases.

In some embodiments, the anti-CD45RC antibody for use according to the invention is used to prevent and/or treat monogenic diseases involving a gene not associated with immune function but whose deficiency is associated with inflammation and/or immune reactions.

Examples of such monogenic diseases involving a gene not associated with immune function but whose deficiency is associated with inflammation and/or immune reactions include, but are not limited to, Duchenne muscular dystrophy (DMD), cystic fibrosis, lysosomal storage diseases and α1-anti-trypsin deficiency.

In one embodiment, the monogenic disease involving a gene not associated with immune function but whose deficiency is associated with inflammation and/or immune reactions is DMD.

In some embodiments, the anti-CD45RC antibody for use according to the invention is used to prevent and/or treat monogenic diseases involving a gene involved in the immune system and whose deficiency generates inflammation and/or autoimmune reactions.

Examples of such monogenic diseases involving a gene involved in the immune system and whose deficiency generates inflammation and/or autoimmune reactions include, but are not limited to, T-cell primary immunodeficiencies (such as, e.g., such as immunodysregulation polyendocrinopathy enteropathy X-linked syndrome [IPEX] and autoimmune polyendocrinopathy-candidiasis-ectodermal dystrophy [APECED]), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

In one embodiment, the monogenic disease involving a gene involved in the immune system and whose deficiency generates inflammation and/or autoimmune reactions is APECED.

In one embodiment, the anti-CD45RC antibody for use according to the invention is used to prevent and/or treat monogenic diseases selected from diseases involving:
genes not associated with immune function but whose deficiency is associated with inflammation and/or immune reactions, such as genes deficient in the following diseases: Duchenne muscular dystrophy (DMD), cystic fibrosis, lysosomal diseases and α1-anti-trypsin deficiency; and/or
genes involved in the immune system and whose deficiency generates inflammation and/or autoimmune reactions, such as genes deficient in the following diseases: T-cell primary immunodeficiencies (such as, e.g., such as immunodysregulation polyendocrinopathy enteropathy X-linked syndrome [IPEX] and autoimmune polyendocrinopathy-candidiasis-enteropathy dystrophy [APECED]), B cell primary immunodeficiencies, Muckle-Wells syndrome, mixed autoinflammatory and autoimmune syndrome, NLRP12-associated hereditary periodic fever syndrome and tumor necrosis factor receptor 1 associated periodic syndrome.

In one embodiment, the anti-CD45RC antibody for use according to the invention is used to prevent and/or treat DMD and/or APECED.

In a further embodiment, the anti-CD45RC antibody for use according to the invention is used to reduce, alleviate, lessen or inhibit symptoms and/or signs associated with monogenic diseases.

Symptoms and signs associated with monogenic diseases in which autoimmune response and/or inflammation is/are involved include, but are not limited to, weight loss, weakness, fatigue, low-grade fever, pain in muscles, muscles, joints, diarrhea, diabetes, hormonal changes, alopecia, skin depigmentation, and deteriorated tissue architecture of different tissues with lymphocyte infiltrates.

In particular, monogenic symptoms have been showed to be associated with several molecular dysregulations, including, but not limited to, increased frequency of CD45RC⁺ cells (particularly T CD45RC⁺ cells), decreased frequency of CD45RC⁻ cells (particularly T, B and NK CD45RC⁻ cells), increased frequency of T CD4+ and CD8⁺ effectors, and reduction of FoxP3⁺ T_{regs}.

In some embodiments, the antibody for use according to the invention induces immune tolerance in a subject. In some embodiments, the antibody for use according to the invention induces immune tolerance in a subject.

In some embodiments, the antibody for use according to the invention mediates depletion of T cells expressing CD45RC on a high or intermediary level.

The relative level of expression of CD45RC can be measured using techniques known to the skilled artisan, including, but not limited to, cytofluorometry. Three types of cells can be distinguished: cells presenting a high level (CD45RC^{high}), an intermediary level (CD45RC^{int}) and a negative level (CD45RC^{neg}) of CD45RC expression, as illustrated in FIG. 9. As used herein, cells designated as “CD45RC⁺” encompass CD45RC^{high} and CD45RC^{int} cells.

In one embodiment, the anti-CD45RC antibody for use according to the present invention is an anti-CD45RC antibody that depletes T CD45RC^{high} cells. T CD45RC^{high} cells are T lymphocytes that express the CD45RC marker (CD45RC⁺) in high quantity, as defined above. It is understood that said antibody that depletes T CD45RC^{+high} cells may be able to deplete other types of CD45RC⁺ cells, such as NK or B CD45RC⁺ cells.

As used herein, the terms “deplete” or “depleting”, with respect to cells expressing CD45RC, refer to a measurable decrease in the number of cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the terms refer to a decrease in the number of CD45RC⁺ cells in a subject or in a sample to an amount below detectable limits. According to the present invention, the anti-CD45RC antibody for use according to the present invention specifically mediates depletion of effector cells strongly expressing CD45RC, in particular those designed as CD45RC^{high} T_{eff}.

In particular, said anti-CD45RC antibody for use according to the present invention depletes CD45RC^{high} T cells by binding to hCD45RC and transducing pro-apoptotic signals and/or by activating antibody-dependent cell mediated cytotoxicity (ADCC), complement dependent cytotoxicity (CDC) and/or antibody-dependent phagocytosis.

In some embodiments, the anti-CD45RC antibody for use according to the present invention mediates antibody-dependent cell mediated cytotoxicity (ADCC).

In some embodiments, the anti-CD45RC antibody for use according to the present invention mediates complement dependent cytotoxicity (CDC).

In some embodiments, the anti-CD45RC antibody for use according to the present invention mediates antibody-dependent phagocytosis.

In a particular embodiment, the isolated antibody or binding fragment thereof according to the present invention may be conjugated to a cytotoxic agent or a growth inhibitory agent.

In some embodiments, the antibody for use according to the invention is able to expand and/or potentiate regulatory T cells in a subject.

As used herein, the term “expand” refers to the process of converting and/or amplifying a given population of cells (e.g., immune cells such as T_{regs}). As used herein, the term “potentiate” refers to the process of increasing the function of a given population of cells (e.g., increasing the suppressive capacity of T_{regs} cells).

“Regulatory T cells” or “T_{regs}” are T cells that suppress an abnormal or excessive immune response and play a role in immune tolerance. T_{regs} are typically “forkhead box P3 (Foxp3⁺) regulatory T cells” and/or “CD45RC^{low/-} cells”. As used herein, the terms “forkhead box P3 (Foxp3⁺) regulatory T cells” and “CD45RC^{low/-} cells” refer to 0.1-10% of CD4⁺ and CD8⁺ T cells in humans and rodents whose characteristic marker is the transcription factor Foxp3.

In some embodiments, the antibody for use according to the invention is able to expand and/or potentiate Foxp3⁺ and/or CD45RC^{low} T_{regs}.

In one embodiment, the anti-CD45RC antibody for use according to the invention is to be administered in the form of a pharmaceutical composition, comprising said anti-CD45RC antibody and a pharmaceutically acceptable carrier or excipient or vehicle.

The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient or vehicle refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, oral, parenteral, intranasal, intravenous, intramuscular, subcutaneous administration and the like.

In one embodiment, the pharmaceutical composition comprises an anti-CD45RC monoclonal antibody selected from the group comprising or consisting of OX-22, OX-32, 3H1437, MT2, RP1/12, DNL 1.9, C455.1F and derivatives thereof; and a pharmaceutically acceptable carrier or excipient or vehicle.

Therefore, an antibody of the invention may be combined with pharmaceutically acceptable excipients carrier or vehicle, and optionally sustained-release matrices, such as biodegradable polymers, to form therapeutic compositions.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment. To prepare pharmaceutical compositions, an effective amount of the antibody may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. in all cases, the form must be sterile and must be fluid to the extent that easy syringability exists. It must be stable under the conditions of manufacture and storage and must be preserved against the contaminating action of microorganisms, such as bacteria and fungi.

In one embodiment, the anti-CD45RC antibody for use according to the invention will be formulated for administration to the subject.

In one embodiment, the anti-CD45RC antibody for use according to the invention is to be administered systemically or locally.

In one embodiment, the anti-CD45RC antibody for use according to the invention is to be administered by injection, orally, topically, nasally, buccally, rectally, vaginaly, intratracheally, by endoscopy, transmucosally, or by percutaneous administration.

In one embodiment, the anti-CD45RC antibody for use according to the invention is to be injected, preferably systemically injected.

Examples of formulations adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

Examples of systemic injections include, but are not limited to, intravenous (iv), subcutaneous, intramuscular (im), intradermal (id), intraperitoneal (ip) injection and perfusion.

In one embodiment, when injected, the anti-CD45RC antibody for use according to the invention is sterile. Methods for obtaining a sterile composition include, but are not limited to, GMP synthesis (where GMP stands for “Good manufacturing practice”).

Sterile injectable forms of a composition may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

It will be understood that other suitable routes of administration are also contemplated in the present invention, and the administration mode will ultimately be decided by the attending physician within the scope of sound medical judgment. Apart from administration by injection (iv, ip, im and the like), other routes are available, such as nebulization (Respaud et al., 2014. MAbs. 6(5):1347-55; Guilleminault et al., 2014. J Control Release. 196:344-54; Respaud et al., 2015. Expert Opin Drug Deliv. 12(6):1027-39) or subcutaneous administration (Jackisch et al., 2014. Geburtshilfe Frauenheilkd. 74(4):343-349; Solal-Celigny, 2015. Expert Rev Hematol. 8(2):147-53).

In one embodiment, the anti-CD45RC antibody for use according to the invention is to be administered to the subject in need thereof in a therapeutically effective amount.

It will be however understood that the total daily usage of the anti-CD45RC antibody for use according to the invention will be decided by the attending physician within the scope of sound medical judgment. The specific therapeutically effective dose level for any particular patient will depend upon a variety of factors including the disease being treated and the severity of the disease; activity of the anti-CD45RC antibody employed; the age, body weight, general health, sex and diet of the subject; the time of administration, route of administration, and rate of excretion of the specific the anti-CD45RC antibody employed; the duration of the treatment; drugs used in combination or coincidental with the specific the anti-CD45RC antibody employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose. However, the daily dosage of the antibodies may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. A therapeutically effective amount of the drug is ordinarily supplied at a dosage level from 0.0002 mg/kg to about 20 mg/kg of body weight per day, especially from about 0.001 mg/kg to 10 mg/kg of body weight per day.

In one embodiment, a therapeutically effective amount of the anti-CD45RC antibody for use according to the invention ranges from about 0.1 mg/kg to about 5 mg/kg, from about 0.2 mg/kg to about 4 mg/kg, from about 0.3 mg/kg to about 3 mg/kg, from about 0.4 mg/kg to about 2.5 mg/kg, from about 0.5 mg/kg to about 2 mg/kg.

In one embodiment, a therapeutically effective amount of the anti-CD45RC antibody for use according to the invention ranges from about 10 μg/kg to about 400 μg/kg, from about 20 μg/kg to about 300 μg/kg, from about 30 μg/kg to about 250 μg/kg, from about 35 μg/kg to about 200 μg/kg, from about 40 μg/kg to about 160 μg/kg.

In one embodiment, a therapeutically effective amount of the anti-CD45RC antibody for use according to the invention is to be administered once a day, twice a day, three times a day or more.

In one embodiment, a therapeutically effective amount of the anti-CD45RC antibody for use according to the invention is to be administered every day, every two days, every three days, every four days, every five days, every six days.

In one embodiment, a therapeutically effective amount of the anti-CD45RC antibody for use according to the invention is to be administered every week, every two weeks, every three weeks.

In one embodiment, a therapeutically effective amount of the anti-CD45RC antibody for use according to the invention is to be administered every month, every two months, every three months, every four months, every five months, every six months.

In a preferred embodiment, a therapeutically effective amount of the anti-CD45RC antibody for use according to the invention is to be administered every 12 hours, every 24 hours, every 36 hours, every 48 hours, every 60 hours, every 72 hours, every 84 hours, every 96 hours.

In a preferred embodiment, a therapeutically effective amount of the anti-CD45RC antibody for use according to the invention is to be administered every 60 hours. In a preferred embodiment, a therapeutically effective amount of the anti-CD45RC antibody for use according to the invention is to be administered every 84 hours.

In one embodiment, the anti-CD45RC antibody for use according to the invention is for acute administration. In one embodiment, the anti-CD45RC antibody for use according to the invention is for chronic administration.

In one embodiment, a therapeutically effective amount of the anti-CD45RC antibody for use according to the invention is to be administered for about 5 days, 7 days, 10 days, 14 days, 21 days, 28 days, 1 month, 2 months, 3 months, 6 months, 1 year or more.

In one embodiment, a therapeutically effective amount of the anti-CD45RC antibody for use according to the invention is to be administered for a period of time ranging from about one week to about eight weeks, from about two weeks to about seven weeks, from about two weeks to about six weeks, from about two weeks to about five weeks.

In a preferred embodiment, a therapeutically effective amount of the anti-CD45RC antibody for use according to the invention is to be administered for a period of time ranging from about 10 days to about 40 days, from about 15 days to about 35 days, from about 20 days to about 30 days.

In one embodiment, the anti-CD45RC antibody for use according to the invention is to be administered alone.

In another embodiment, the anti-CD45RC antibody for use according to the invention is to be administered in combination with at least one therapeutic drug, such as, e.g., before, concomitantly with or after a therapeutic drug.

In some embodiments, the therapeutic drug may be an immunosuppressive and/or an anti-inflammatory drug. The term “immunosuppressive and/or anti-inflammatory drug” relates to a class of drugs that suppress, reduce, lessen or alleviate the strength of immune response in a subject. Such drugs are particularly suitable for treating monogenic diseases linked to genes involved in the immune system or to genes not associated with immune functions but whose deficiency is associated with inflammation and/or immune reactions. The use of immunosuppressive drug according to the invention, along with the anti-CD45RC antibody according to the invention, would therefore help to reduce the impact of the immune response in the subject affected with a monogenic disease.

Suitable examples of immunosuppressive drugs include, without limitation, mTOR inhibitors such as, e.g., sirolimus, everolimus, ridaforolimus, temsirolimus, umirolimus and zotarolimus; IL-1 receptor antagonists such as, e.g., anakinra; antimetabolites such as, e.g., azathioprine, leflunomide, methotrexate, mycophenolic acid and teriflunomide; IMiDs such as, e.g., apremilast, lenalidomide, pomalidomide and thalidomide; and antibodies such as, e.g., eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, zolimomab aritox, atorolimumab, cedelizumab, fontolizumab, maslimomab, morolimumab, pexelizumab, reslizumab, rovelizumab, siplizumab, talizumab, telimomab aritox, vapaliximab, vepalimomab, abatacept, belatacept, etanercept, pegsunercept, aflibercept, alefacept and rilonacept.

Suitable examples of anti-inflammatory drug include, without limitation, corticoids (such as glucocorticoids selected from dexamethasone, betamethasone, betamethasone-17-valerate, triamcinolone, triamcinolone acetonide, fluocinolone acetonide, fluocinonide, cortisone, hydrocortisone, prednisone, methylprednisolone, desonide, budesonide and deflazacort).

Preferably, the immunosuppressive and/or an anti-inflammatory drug is prednisolone or deflazacort.

Accordingly, the present invention encompasses pharmaceutical compositions comprising an anti-CD45RC antibody as previously defined, an immunosuppressive and/or anti-inflammatory drug and a pharmaceutically acceptable carrier or excipient or vehicle. In some embodiments, the pharmaceutical composition comprises an anti-CD45RC antibody as previously defined, prednisolone or deflazacort, and a pharmaceutically acceptable carrier or excipient or vehicle.

In another embodiment, the anti-CD45RC antibody for use according to the invention is to be administered in combination with gene therapy or cell therapy. Advantageously, said gene therapy or cell therapy is used before or after the use of said anti-CD45RC antibody, preferentially before the use of said anti-CD45RC antibody.

Accordingly, the present invention encompasses pharmaceutical compositions comprising an anti-CD45RC antibody as previously defined, an adeno-associated virus or lentivirus containing at least one mini-dystrophin gene and a pharmaceutically acceptable carrier or excipient or vehicle. In some embodiments, the pharmaceutical composition is a combined preparation for simultaneous, separate or sequential use. The term “mini-dystrophin gene” refers herein to 6- to 8-kb synthetic mini-dystrophin genes such as the mini-dystrophin genes described by Odom et al. (2011. Mol Ther. 19(1):36-45), Clemens et al. (1995. Hum Gene Ther. 6(11):1477-85), Harper et al. (2002. Nat Med. 8(3):253-61) and Lai et al. (2009. J Clin

Invest. 119(3):624-35).

In another embodiment, the anti-CD45RC antibody for use according to the invention is to be administered in combination with both an immunosuppressive and/or anti-inflammatory drug, and with gene therapy or cell therapy.

Accordingly, the present invention encompasses pharmaceutical compositions comprising an anti-CD45RC antibody as previously defined, an immunosuppressive and/or anti-inflammatory drug, an adeno-associated virus or lentivirus containing at least one mini-dystrophin gene and a pharmaceutically acceptable carrier or excipient or vehicle. In some embodiments, the pharmaceutical composition is a combined preparation for simultaneous, separate or sequential use.

### BRIEF DESCRIPTION OF THE DRAWINGS

FIG. 1 is a set of graphs showing the number of leukocytes in muscle and spleen of Dmd^{mdx} rats. Hind limb skeletal muscles and spleen were harvested from littermate wild-type (WT) or Dmd^{mdx} (KO) rats at the indicated time points of age. Muscle and spleen were digested with collagenase and mononuclear cells were isolated using a density gradient. (A) Representative dot-plot analysis of mononuclear cells muscle (left panel) and spleen (right panel) from animals at 8 weeks of age stained with a viability dye and a pan anti-leukocyte CD45 monoclonal antibody (OX1). (B) Number of CD45⁺ cells by gram of muscle tissue or by total spleen at different time points. WT, n=4, 5, 7, 7, 9 at 2, 4, 8, 12 and 16 weeks, respectively. KO, n=3, 6, 10, 11, 16 at 2, 4, 8, 12 and 16 weeks, respectively. * p<0.05, ***p<0.001, ****p<0.0001.

FIG. 2 is a set of two graphs showing the number of TCR cells in muscle and spleen of littermate WT and Dmd^{mdx} rats. PBMCs from hind limbs (left panel) and spleen (right panel) were obtained as explained in the legend of FIG. 1. The number of TCR⁺ cells was determined by staining PBMCs with an anti-TCRab monoclonal antibody (clone R7/3) and calculating the number of TCR cells among viable CD45R⁺ cells. * p<0.05, **p<0.01.

FIG. 3 is a set of four graphs showing the numbers of T CD8⁺CD45RC^{high} and CD45RC^{int/neg} cells after treatment using an anti-CD45RC monoclonal antibody. Control littermate wild-type (WT) or Dmd^{mdx} (KO) rats received from week 2 of age intraperitoneal injections of the mouse anti-rat CD45RC monoclonal antibody up to week 12, when the animals were sacrificed. Muscle and spleen mononuclear cells were isolated and analyzed for the presence of TCR⁺CD8⁺ cells expressing CD45RC at either high (CD45RC^{high}) or intermediate/negative levels (CD45RC^{int/neg}). Each point represents a single animal * p<0.05, ***p<0.001.

FIG. 4 is a graph showing the muscle strength in Dmd^{mdx} rats after treatment with an anti-CD45RC monoclonal antibody. Wild type (WT) or Dmd^{mdx} (KO) rats received intraperitoneal injections of the mouse anti-rat CD45RC or prednisolone from week 2 of age up to week 12, when the muscle strength was analyzed using a grip test. Each point represents a single animal. * p<0.05.

FIG. 5 is a set of four graphs showing the numbers of T CD8+CD45RC^{high} and CD45RC^{int/neg} cells after treatment using prednisolone. Control littermate wild-type (WT) or Dmd^{mdx} (KO) rats received from week 2 of age intraperitoneal injections of prednisolone up to week 12, when the animals were sacrificed. Muscle and spleen mononuclear cells were isolated and analyzed for the presence of TCR⁺CD8⁺ cells expressing CD45RC at either high (CD45RC^{high}) or intermediate/negative levels (CD45RC^{int/neg}). Each point represents a single animal. * p<0.05.

FIG. 6 is a set of two graphs showing the weight of wild-type or Dmd^{mdx} rats during treatment using prednisolone or anti-CD45RC monoclonal antibody. Control littermate wild-type (WT) or Dmd^{mdx} (KO) rats received intraperitoneal injections of prednisolone or anti-CD45RC monoclonal antibody (OX22) from week 2 up to week 12 of age and weight was recorded at the indicated time points.

FIG. 7 is a set of photographs and a graph showing reduced autoimmune signs of disease in anti-CD45RC mAb-treated Aire^{-/-} rats. Aire^{-/-} rats received from week 2 of age intraperitoneal injections of the mouse anti-rat CD45RC MAb (clone 0X22, 1.5 mg/kg twice per week) up to week 20, when the animals were sacrificed. (A). Picture showing visual aspect of 20 weeks old Aire^{-/-} rats treated with isotype control (top row) or anti-CD45RC mAb (bottom row). (B) Picture showing the size of the thymus from anti-CD45RC mAb or isotype control Aire^{-/-} treated rats. (C) Weights of anti-CD45RC mAb or isotype control Aire^{-/-} littermate SPD rats were measured every week during 20 weeks following birth. Results are shown as the % of initial body weight starting at week 2 after birth (mean 31.3 g)±SEM (n=3).

FIG. 8 is a set of graphs showing efficient depletion of CD45RC^{high} T cells in spleen and mesenteric lymph nodes (MLN) following anti-CD45RC mAb administration. Representative dot-plot analysis of mononuclear cells (CD4⁺ and CD8⁺ T cells) from MLN and spleen from Aire^{-/-} treated with anti-CD45RC or isotype control Mabs or WT untreated animals at 20 weeks of age and treated from week 2 stained with a viability dye and a pan anti-leukocyte CD45 MAb (OX1), CD3, TCR, CD4 and CD45RC MAb.

FIG. 9 is a graph defining 3 populations of CD8⁺ cells according to the different levels of CD45RC expression (CD45RC^{high}, CD45RC^{int} and CD45RC^{neg}) in rat CD8+ T cells labeled with the anti-CD45RC Mab OX22.

FIG. 10 is a set of two SDS-PAGE gels showing the effect of anti-CD45RC MAb treatment on serum autoantibodies in Aire-deficient rats. Aire-deficient rats were treated with (A) an isotype control MAb or (B) an anti-CD45RC MAb (OX22) at 1.5 mg/kg twice per week from weeks 2 to 20 of age. Silver staining. β-actin was used as a control.

FIG. 11 is a set of eight photographs of tissue sections (thymus, pancreas, skin, kidney) stained with hematoxilin-eosine-safran, showing the tissue architecture and lymphocyte infiltrates in rats treated with an isotype antibody (left photographs) or with an anti-CD45RC antibody (right photographs). White arrows represent major regions of each organ differing between anti-CD45RC or isotype treated recipients.

### EXAMPLES

Materials and Methods Related to Examples 1 to 4

### Preparation of Muscle and Spleen Single-Cell Suspensions

Muscles of both hind limbs from WT or Dmd^{mdx} rats were excised and weighed. Muscles were minced and placed in gentle MACS C tubes with collagenase D in the presence of FCS 2%, 1 mM EDTA. Two runs of 30 minutes each in a gentle MACS dissociator were performed with new collagenase added between each run. Cells were suspended in 30 mL of PBS FCS 2%, 1 mM EDTA, were then applied to 15 mL of Hystopaque and centrifuged at 1000 g for 30 minutes without a break. Mononuclear cells were collected from the Hystopaque and PBS interface, washed and suspended in PBS FCS 2%, 1 mM EDTA.

Spleen was harvested, perfused with PBS and digested by collagenase D for 15 minutes at 37° C. Cells were suspended in PBS FCS 2%, 1 mM EDTA and mononuclear cells were recovered as explained above.

### Flow Cytometry Analysis

Mononuclear cells were stained with antibodies against the following antigens: CD45 (clone OX-1), T-cell receptor (TCR; clone R7/3), CD45RC (clone OX22), CD8 (clone OX8) and CD4 (W3/25), as well as with viability dyes eFluor506 or eFluor450, all from eBiosciences. Analysis was performed on a BD FACS Verse with FACSuite Software version 1.0.6. Post-acquisition analysis was performed with FlowJo software.

### Treatment with Anti-CD45RC or Prednisolone

Wild-type (WT) or Dmd^{mdx} (KO) rats received intraperitoneal injections of a mouse anti-rat CD45RC monoclonal antibody (clone OX22, 2 mg/kg, every 3.5 days) from week 2 of age up to week 12.

Prednisolone was administered by intraperitoneal injections (0.5 mg/kg, 5 days a week) from week 2 of age up to week 12.

At week 12 of age treated rats were analyzed for muscle strength using a grip test.

### Grip Test

Rats were placed with their forepaws on a grid and were gently pulled backward until they released their grip. A grip meter (Bio-GT3, BIOSEB, France), attached to a force transducer, measured the peak force generated.

### Example 1

Analysis of Total Keukocytes and T cells in Dmd^{mdx} Rats

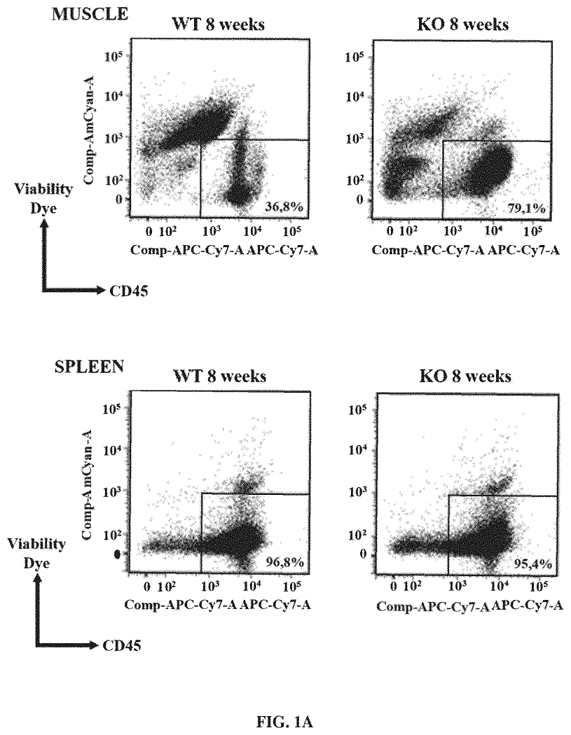

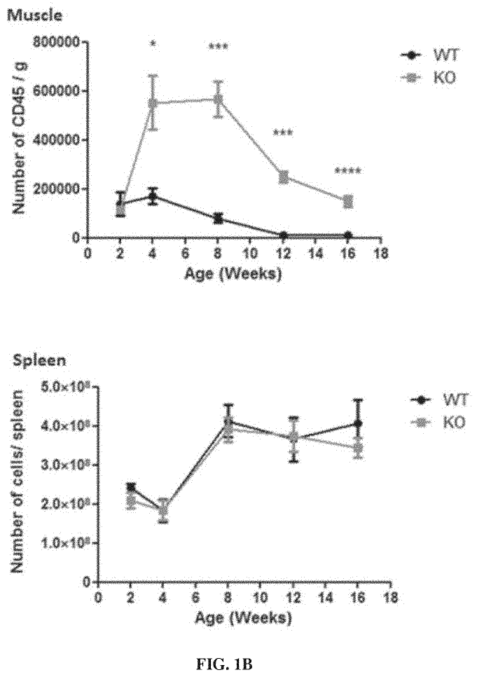

Leukocytes in the muscle and spleen of Dmd^{mdx} rats were analyzed by flow cytometry (FIG. 1).

Total leukocytes in the muscle of littermate WT and Dmd^{mdx} rats were comparable at 2 weeks of age, but at 4 weeks, Dmd^{mdx} rats showed a sharp increase that was maintained until week 8 and then decreased at weeks 12 and 14, although still significantly higher than in littermate WT rats.

Spleen leukocyte numbers were comparable between WT and Dmd^{mdx} rats at all-time points analyzed.

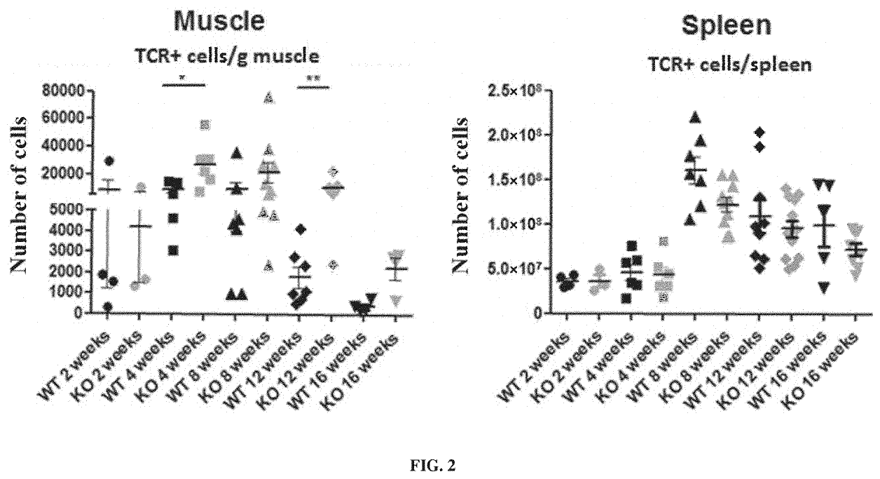

Analysis of the number of TCR cells in muscle and spleen of littermate WT and Dmd^{mdx} rats showed a significant increase in the muscle of Dmd^{mdx} rats at 4 and 12 weeks of age with an increased tendency at week 8 and 16 weeks (FIG. 2).

The numbers of TCR⁺ cells in spleen did not show differences between WT and Dmd^{mdx} rats at any time point.

### Example 2

Treatment with Anti-CD45RC Monoclonal Antibody Depletes CD45RC^{high} T Cells

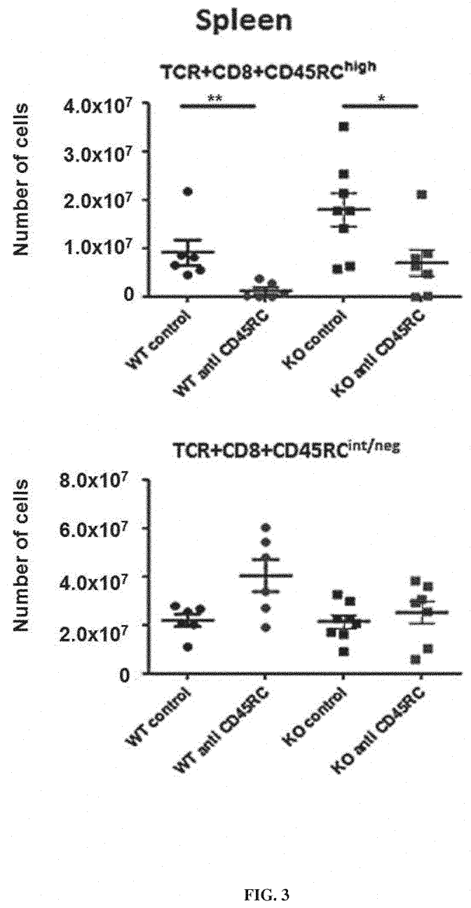

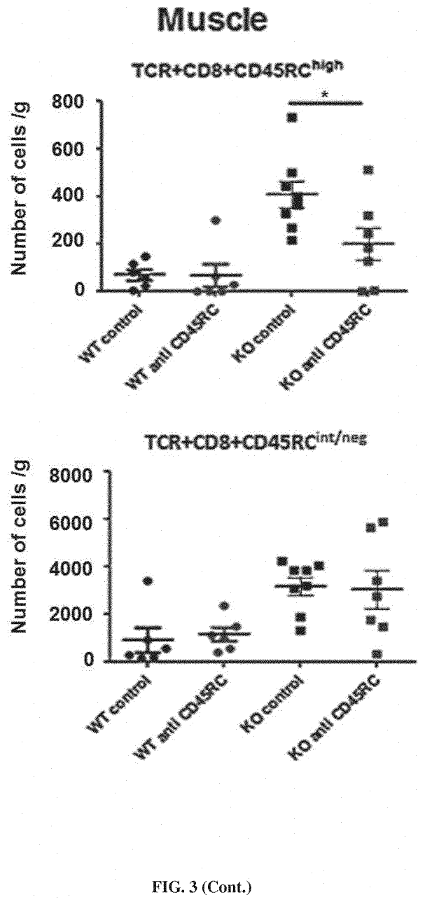

Administration of a mouse anti rat-CD45RC monoclonal antibody from week 2 of age resulted in partial depletion of T CD8⁺ CD45RC^{high} cells analyzed at 12 weeks of age in muscle of Dmd^{mdx} rats and in spleen of both littermate WT and Dmd^{mdx} rats (FIG. 3).

T CD8⁺ or CD4⁺ CD45RC^{int/neg}, including all CD8⁺ and CD4⁺T_{regs}, were not modified in the spleen or muscle of neither WT nor Dmd^{mdx} rats (FIG. 3).

All other major leukocyte populations (macrophages, B cells and NK cells) were unchanged (data not shown).

### Example 3

Treatment with Anti-CD45RC Monoclonal Antibody Improves Muscle Strength in Dmd^{mdx} Rats

To examine whether muscle function was improved by the anti-CD45RC monoclonal antibody treatment, muscle strength was analyzed by a grip test of forelimbs in WT and Dmd^{mdx} rats at 12 weeks of age after initiation of treatment at 2 weeks of age.

A significant decrease in forelimb grip strength indicated a generalized alteration in the whole-body muscular performance. As previously described by Larcher et al. (2014. PLoS One. 9(10):e110371), a 30% weaker force was exerted by Dmd^{mdx} rats compared to WT littermates.

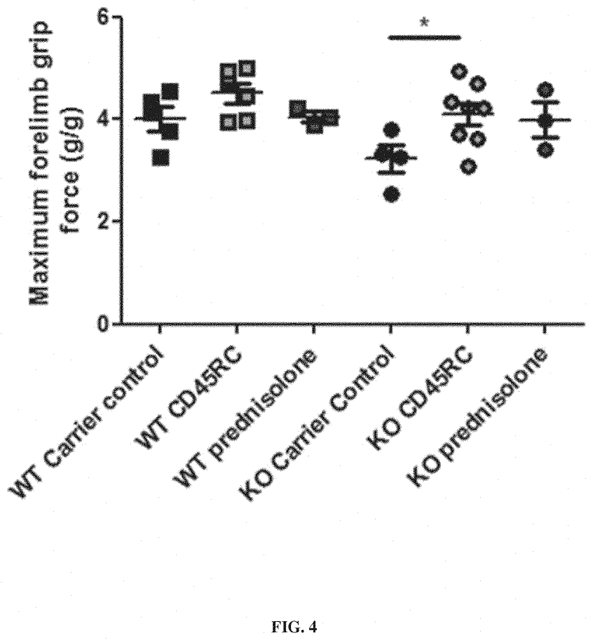

After administration of the anti-CD45RC monoclonal antibody, muscle strength in Dmd^{mdx} rats vs. untreated Dmd^{mdx} rats was significantly increased and was indistinguishable from WT littermate controls (FIG. 4).

### Example 4

Treatment with Prednisolone Improved Skeletal Muscle Strength Since corticoids are standard treatment in DMD patients (Alman, 2005. J Pediatr Orthop. 25(4):554-6), the effect of prednisolone was analyzed in the muscle strength of Dmd^{mdx} rats.

Treatment of Dmd^{mdx} rats with prednisolone, since 2 weeks of age, increased muscle strength at 12 weeks to levels identical to those of WT or anti-CD45RC-treated rats (FIG. 4).

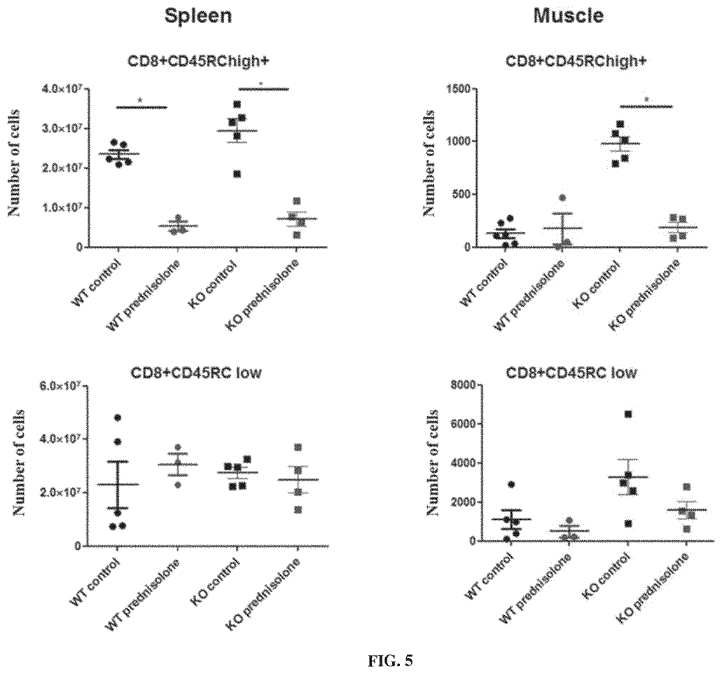

Interestingly, prednisolone-treated rats also showed a specific decrease of CD8⁺CD45RC^{high} cells in both muscle and spleen of Dmd^{mdx} rats and in spleen of WT rats, whereas CD8⁺CD45RC^{int/neg} cells were maintained (FIG. 5).

Similarly, CD4⁺CD45RC^{high} cells were numerically reduced in both muscle and spleen of Dmd^{mdx} rats and in spleen of WT rats whereas CD8⁺CD45RC^{int/neg} cells were maintained (data not shown).

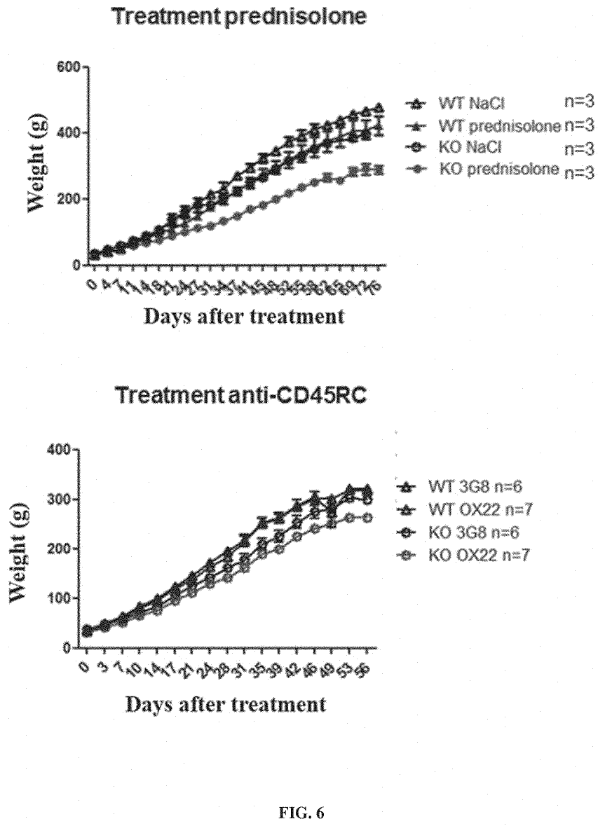

Prednisolone-treated Dmd^{mdx} rats showed severe reduction in animal growth whereas anti-CD45RC monoclonal antibody-treated rats did not (FIG. 6).

### Example 5

Treatment of APECED Disease

### Materials and Methods

### Cell Isolation

Spleen and lymph nodes were digested by collagenase D for 30 minutes at 37° C. The reaction was stopped by adding 0.01 mM EDTA.

Cells from blood and bone marrow were also isolated and red blood cells were lysed using a lysis solution (8,29 g NH₄Cl, 1 g KHCO3, 37,2 mg EDTA qsp 1 L deionized water pH 7.2-7.4).

Antibodies and Flow Cytometry

Cellular phenotype was analyzed using monoclonal antibodies from BD pharmigen: against TCRαβ (R73), CD25 (Ox39), CD4 (Ox35), and CD45 (Ox1).

Abs against CD45RC (Ox22) and CD8 (Ox8) produced in our lab were used.

Antibodies were used to stain cells and fluorescence was measured with a FACSCanto II flow cytometer (BD Bioscience) and FlowJo software was used to analyze data. Cells were first gated on their morphology and then dead cells were excluded by staining with fixable viability dye efluor 506 (Ebioscience).

### Treatment with Anti-CD45RC or Isotype Control

Aire^{-/-} (KO) rats received intraperitoneal injections of a mouse anti-rat CD45RC monoclonal antibody (clone OX22, 1.5 mg/kg, every 3.5 days) from week 2 of age up to week 20 when the animals were sacrificed. Isotype control was administered similarly. The rats were weighted twice a week from the start of the treatment until the day of sacrifice. At week 20 of age, treated rats were sacrificed and analyzed.

### Autoantibodies Detection

Serum from Aire-deficient rats treated with anti-CD45RC or treated with an isotype control at 20 weeks of age were incubated with membranes in which lysates from different organs (spleen, colon, kidney, eye, lung, testis, mesenteric lymph nodes [MLN] and ileum) from an Il2rg-deficient rat (Il2rg: interleukin 2 receptor subunit gamma), thus without endogenous immunoglobulins, were previously electrophoresed. Binding of rat autoantibodies was revealed using anti-rat immunoglobulin antibodies or with an anti-β-actin antibody coupled to peroxidase.

### Histochemistry

Organs from Aire-deficient rats treated with anti-CD45RC or treated with an isotype control were harvested at 20 weeks of age. Tissue sections (thymus, pancreas, skin, kidney) were prepared and stained with hematoxilin-eosine-safran.

### Results

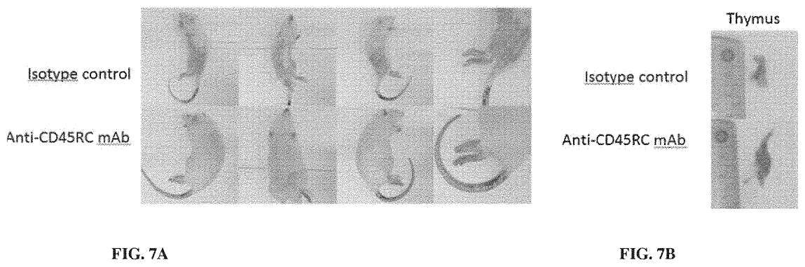

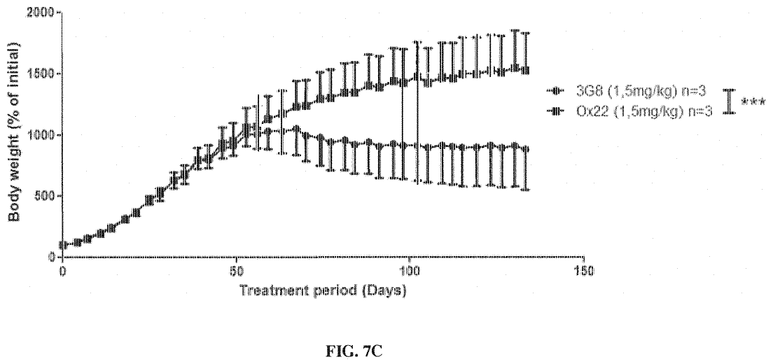

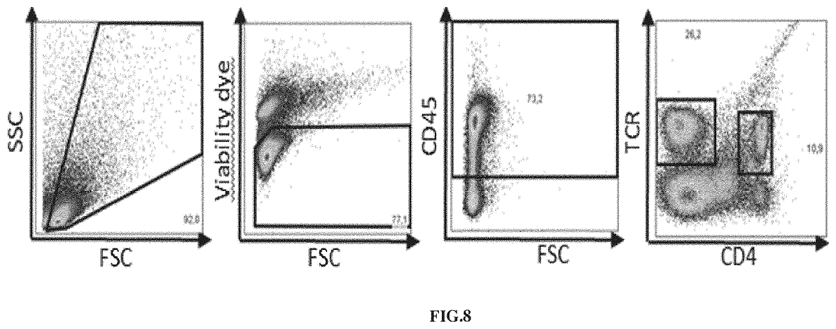

We observed that 100% of Aire-deficient rats spontaneously developed alopecia and skin depigmentation in isotype control treated animals (FIG. 7A, top row), signs that correlate with a severe auto-immune disease and that are clinical manifestations regularly found in APECED patients.

In contrast, Aire-deficient rats treated with anti-CD45RC mAb did not develop alopecia and skin depigmentation (FIG. 7A, bottom row), had a bigger thymus size (FIG. 7B) and a normal body weight (FIG. 7C), indicating a normal growth, a preserve thymus structure and altogether a decreased auto-immune disease.

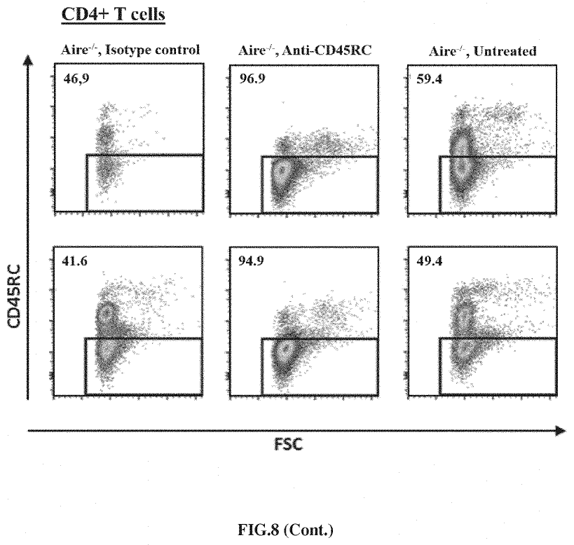

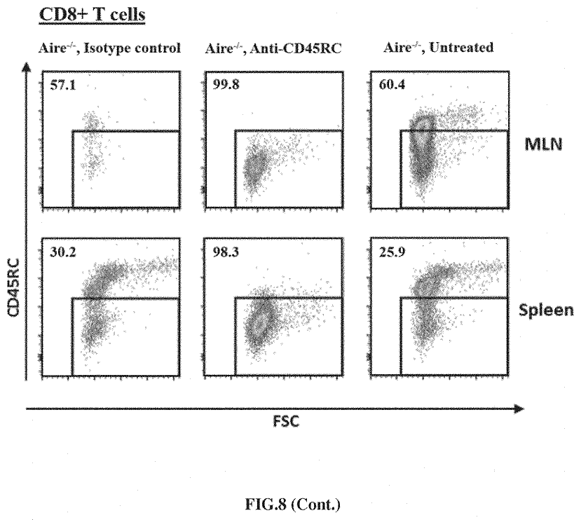

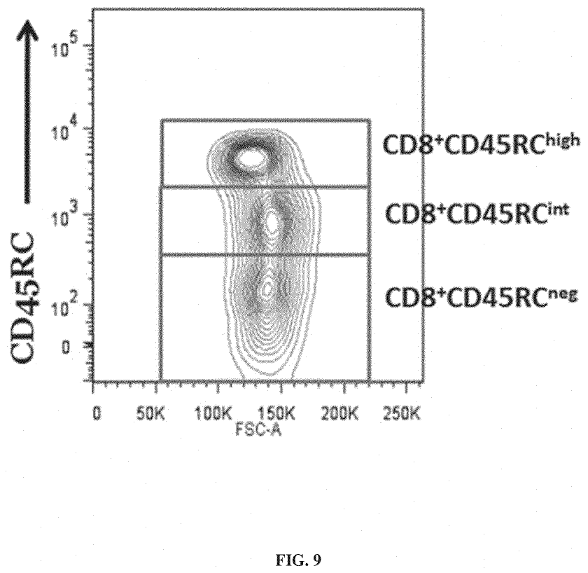

Administration of a mouse anti rat-CD45RC monoclonal antibody from week 2 of age resulted in strong depletion of T CD8⁺ and CD4⁺ CD45RC^{high} cells in both spleen and lymph node in Aire−/− rats compared to rats treated with isotype control and untreated WT rats (FIG. 8).

Numbers of other major leukocyte populations (macrophages, B cells and NK cells) were unchanged (data not shown).

Analyses of autoantibodies directed against tissue antigens was also evaluated, by western blot, in tissue homogenates and serum.

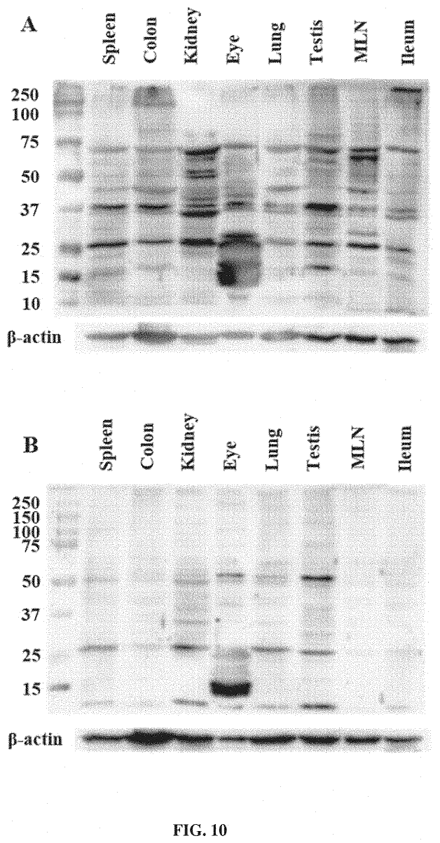

In the absence of anti-rat-CD45RC monoclonal antibody treatment, many bands were detected in different organs, indicating the presence of antoantibodies (FIG. 10A).

By contrast, treatment with an anti-rat-CD45RC monoclonal antibody reduced the number of bands and thus, the number of autoantibodies (FIG. 10B).

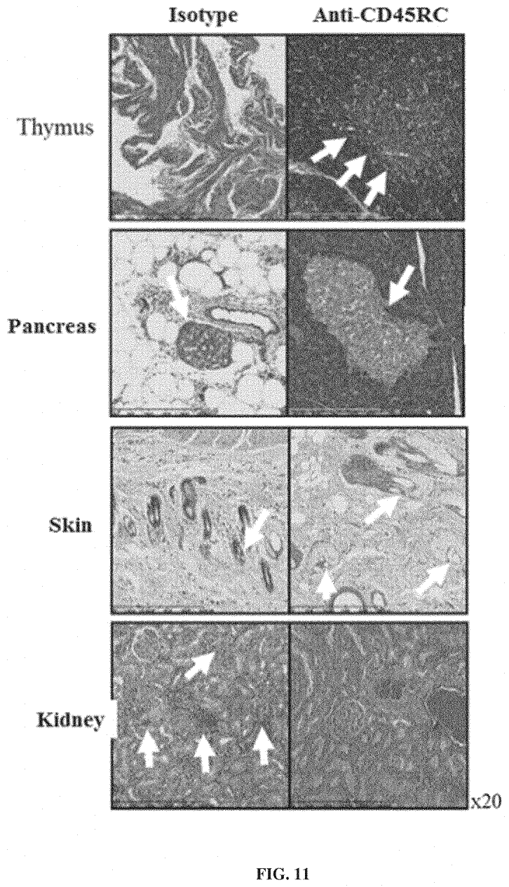

At week 20 of treatment, tissue architecture was destroyed and lymphocyte infiltrates were present in many organs in the absence of treatment, while treatment with the anti-rat-CD45RC monoclonal antibody restored tissue integrity and reduced lymphocyte infiltrates (FIG. 11).

### Conclusion

Altogether, the data presented hereinabove in Examples 1-5 provide a clear demonstration of reduced if not inhibited inflammation and autoimmune reactions typically associated with certain monogenic diseases through the use of anti-CD45RC antibodies. A disbalance between T_{eff} and T_{regs} with the end result of increased T responses, but also B cell-mediated responses through production of autoantibodies, is involved in other monogenic diseases, such as cystic fibrosis, lysosomal diseases, α1-anti-trypsin deficiency, IPEX, B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

1-14. (canceled)

15. A method of preventing and/or treating a monogenic disease in a subject in need thereof, comprising administering to said subject an anti-CD45RC antibody, wherein said monogenic disease is selected from the group consisting of:
a. monogenic diseases caused by a gene which is not associated with immune function but whose deficiency is associated with inflammation and/or immune reactions, and selected from Duchenne muscular dystrophy (DMD), cystic fibrosis, lysosomal diseases and al-anti-trypsin deficiency; and/or
b. monogenic diseases caused by a gene involved in the immune system and whose deficiency generates inflammation and/or autoimmune reactions, and selected from immunodysregulation polyendocrinopathy enteropathy X-linked syndrome (IPEX), autoimmune polyendocrinopathy-candidiasis-ectodermal dystrophy (APECED), B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome.

16. The method according to claim 15, wherein said anti-CD45RC antibody is a monoclonal antibody.

17. The method according to claim 15, wherein said anti-CD45RC antibody is an anti-human CD45RC monoclonal antibody.

18. The method according to claim 15, wherein said anti-CD45RC antibody is a chimeric antibody, a bispecific antibody, a humanized antibody or a fully human antibody.

19. The method according to claim 15, wherein prevention and/or treatment of monogenic diseases comprises reduction, alleviation, lessening and/or inhibition of symptoms or signs associated with said monogenic diseases, preferably of autoimmune and/or inflammatory symptoms or signs.

20. The method according to claim 15, wherein said anti-CD45RC antibody depletes T CD45RC^{high} cells.

21. The method according to claim 15, wherein said anti-CD45RC antibody is a multispecific antibody comprising a first antigen binding site directed against. CDR45RC and at least one second antigen binding site directed against an effector cell able to mediate depletion of T CD45RC^{high} cells through direct binding, antibody-dependent cell-mediated cytotoxicity (ADCC), complement dependent cytotoxicity (CDC), and/or antibody-dependent phagocytosis.

22. The method according to claim 15, wherein said anti-CD45RC antibody is conjugated to a cytotoxic moiety.

23. The method according to claim 15, in the form of a pharmaceutical composition comprising said anti-CD45RC antibody and a pharmaceutically acceptable carrier or excipient or vehicle.

24. The method according to claim 15, wherein said anti-CD45RC antibody is to be administered in combination with an immunosuppressive and/or anti-inflammatory drug.

25. The method according to claim 15, wherein said anti-CD45RC antibody is to be administered in combination with gene therapy or cell therapy.

26. The method according to claim 25, wherein said gene therapy or cell therapy is to be administered before or after administration of said anti-CD45RC antibody, preferentially before administration of said anti-CD45RC antibody.

27. The method according to claim 15, wherein said monogenic disease is selected from DMD, cystic fibrosis, lysosomal storage diseases and al-anti-trypsin deficiency, preferably said monogenic disease is DMD.

28. The method according to claim 15, wherein said monogenic disease is selected from IPEX, APECED, B cell primary immunodeficiencies, Muckle-Wells syndrome, mixed autoinflammatory and autoimmune syndrome, NLRP12-associated hereditary periodic fever syndrome, and tumor necrosis factor receptor 1 associated periodic syndrome, preferably said monogenic disease is APECED.


SEQUENCE LISTING (3 sequences)

SEQ ID NO: 1 - protein, 48 aa, Homo sapiens
  C determinant of hCD45RC
DVPGERSTASTFPTDPVSPLTTTLSLAHHSSAALPARTSNTTITANTS

SEQ ID NO: 2 - DNA, 144 bp, Homo sapiens
  Exon 6 of hCD45RC
gatgtcccaggagagaggagtacagccagcacctttcctacagacccagtttccccattgacaaccaccctcagccttgcacaccacagctctgctgccttacctgcacgcacctccaacaccaccatcacagcgaacacctca

SEQ ID NO: 3 - protein, 1306 aa, Homo sapiens
  hCD45RC
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS